(19) United States
(12) Patent Application Publication
(10) Pub. No.: US 2003/0186946 A1
(43) Pub. Date: Oct. 2, 2003

(54) Suppression of cytotoxic protein conformers

(76) Inventors: Garth James Smith Cooper, Auckland (NZ); Kerry Martin Loomes, Auckland (NZ); Jacqueline Fiona Aitken, Auckland (NZ)

Correspondence Address: Bradford J. Duft, Brobeck, Phleger & Harrison LLP, 12390 El Camino Real, San Diego, CA, 92130, US

(21) Appl. No.: 10/354,893

(22) Filed: Jan. 29, 2003

(30) Foreign Application Priority Data
Jan. 29, 2002 (NZ) S/N 516920

(51) Int. Cl. A61K 31/655; A61K 31/65; A61K 31/542; A61K 31/4745; A61K 31/473; A61K 31/015

(52) U.S. Cl. 514/150; 514/228.2; 514/297; 514/280; 514/152; 514/765

(57) ABSTRACT

Methods of preventing amyloid associated disease comprising preventing protofibril formation using polycyclic compounds related screens and methodologies disclosed.

### REFERENCE TO RELATED APPLICATIONS

This application claims the benefit of New Zealand Provisional Patent Application No. 516920 filed on Jan. 29, 2002. New Zealand Provisional Patent Application No. 516920 is hereby incorporated herein by reference in its entirety.

### FIELD OF THE INVENTION

The present invention relates to polycyclic compounds and to methods for the treatment and prevention of various amyloid-based disease conditions using one or more polycyclic compounds, preferably substituted or unsubstituted polyacene compounds having three to five rings. In a particular aspect, the invention relates to methods of disruption of the transition from a soluble to an insoluble form of amylin. In other aspect, the invention relates to methods of inhibiting aggregation of amyloid pre- or protofibrils and fibrils and inhibiting transition-induced toxicity of amyloid β-fibril and β-sheet formation. This invention provides methods for disrupting islet amyloid in patients with type 2 diabetes mellitus, and for identifying and evaluating polycyclic compounds for use in the prevention or treatment of amyloid-related disease.

### BACKGROUND OF THE INVENTION

All documents referred to herein are incorporated in their entirety by reference, as are all priority applications.

In 1854 Rudolph Virchow introduced and popularized the term amyloid to denote a macroscopic tissue abnormality that exhibited a positive iodine staining reaction. Subsequent light microscopic studies with polarizing optics demonstrated the inherent birefringence of amyloid deposits, a property that increased intensely after staining with Congo red dye. In 1959, electron microscopic examination of ultrathin sections of amyloidotic tissues revealed the presence of fibrils, indeterminate in length and, invariably, 80 to 100 Å in width. Using the criteria of Congophilia and fibrillar morphology, twenty or more biochemically distinct forms of amyloid have been identified throughout the animal kingdom; each is specifically associated with a unique clinical syndrome. Fibrils, also 80 to 100 Å in width, have been isolated from tissue homogenates using differential sedimentation or solubility. X-ray diffraction analysis reveals the fibrils to be ordered in the beta pleated sheet conformation, with the direction of the polypeptide backbone perpendicular to the fibril axis (cross beta structure).

The amyloidoses are a group of pathological conditions in which normally soluble proteins polymerize to form insoluble amyloid fibrils and amyloid deposits. More than 15 proteins form amyloid fibrils currently associated with diverse clinical conditions. Amyloidoses are usually classified into systemic amyloidoses and localized amyloidoses. Systemic amyloidoses (and the proteins which have been thought to cause them in parentheses) include AL amyloidosis (AL amyloid), amyloid A amyloidosis (amyloid A protein), and familial transthyretin amyloidosis (transthyretin). Localized amyloidoses (and the proteins which have been thought to cause them in parentheses) include Alzheimer's disease (amyloid β-peptide), prion diseases (scrapie prion protein), and type 2 diabetes (human amylin).

Amyloid or amyloid proteins refer to a group of diverse extracellular proteins that form amyloid deposits having certain morphological, structural, and chemical properties. Various amyloid deposits have similar affinities for certain dyes and a characteristic appearance under polarized light. Although they vary in amino acid sequence, amyloid proteins found in amyloid deposits consist of aggregations containing interlacing bundles of parallel arrays of fibrils where the protein in the fibrils is organized in a β-pleated sheet structure. The fact that many of the amyloid proteins in amyloid deposits are rich in β-pleated sheet conformation is responsible for the intensely increased birefingence of amyloid fibrils following Congo red staining (Glenner et al., J. Histochem. Cytochem 22:1141-1158 (1974); Glenner and Page, Int. Rev. Exp. Pathol. 15:1-92 (1976); Glenner, N. Engl. J. Med. 302:1283-1292 (Pt. 1) and 133-1343 (Pt. 2) (1980)).

Amyloid fibrils, regardless of the amyloid protein from which they are formed, have been thought to have a cytotoxic effect on various cell types including primary cultured hippocampal neurons (Yankner et al., Science 250:279-282(1990)), pancreatic islet B cells (Lorenzo et al., Nature 368:756-760(1994)) and clonal cell lines (Behl et al., Biochem Biophys. Res. Commun. 186:944-952 (1992); O'Brien et al., Am. J. Pathol. 147:609-616 (1995)). Indeed, only amyloid proteins in fibrillar form have been shown to be cytotoxic (Pike et al., Brain Res. 563:311-314 (1991); Lorenzo and Yankner, Proc. Natl. Acad. Sci. 91:12243-12247 (1994)). It has been hypothesized that the cytotoxic effect of fibrils is mediated by a common mechanism (Lorenzo and Yankner id. (1994); Schubert et al., Proc. Natl. Acad. Sci. USA 92:1989-1993 (1995)). Spontaneous conversion of amyloid peptide from soluble monomer to insoluble fibrillar precipitate may underlie the neurodegeneration associated with Alzheimer's disease. Amyloid deposits of fibrillar human amylin in the pancreas may be a causative factor in type 2 diabetes.

Diabetes mellitus can be defined as a chronic metabolic disorder characterised by elevation of blood glucose (hyperglycaemia), associated with a deficiency in the secretion or action of insulin, and accompanied by chronic vascular complications, which ultimately cause most of the morbidity and mortality (Zimmet et al., Nature, 414:782-787 (2001)). There are two major forms of diabetes, type 1 and type 2. Type 1 diabetes is an autoimmune disorder caused by progressive destruction of the pancreatic β-cells, caused in turn by aberrant cell-mediated immunity. This disease is characterised by an absolute requirement for insulin therapy for survival, and by lymphocytic infiltration of the pancreatic islets during the acute phases of the disease. Type 2 diabetes, on the other hand, is a metabolic disease characterised by the presence of progressive pancreatic islet β-cell failure, the formation of cytotoxic islet a myloid (either soluble or insoluble forms), a nd insulin resistance. These events lead to the progressive failure of regulation of blood glucose, which becomes elevated (hyperglycaemia), and which ultimately leads to complications including diabetic disease of the eyes, kidneys and nerves, and to arterial disease which leads to, inter alia, heart attack, stroke, and gangrene. Although there is no currently agreed molecular basis for these three primary events, peripheral insulin resistance is likely a primary etiological factor that initiates progression of the disease.

In the early stages of type 2 diabetes mellitus, peripheral insulin resistance may be compensated for, by increased insulin output and hyperplasia of the islet β-cells, resulting in only mild symptoms (Bell & Polonsky, Nature, 414:788-791 (2001)). However, increased insulin output also increases the propensity for islet amyloid formation and its subsequent extracellular deposition in the vicinity of the islet β-cells (MacArthur et al., Diabetologia, 42:1219-1227 (1999); Hoppener et al., N. Engl. J. Med., 343:411-419 (2000); Jaikaran & Clark, Biochim Biophys. Acta, 1537:179-203 (2001)).

The occurrence of islet amyloid in type 2 diabetes mellitus was identified over a century ago. Initially, in 1869, Paul Langerhans was the first to describe the endocrine pancreas and how bundled cells appeared to be suspended and unconnected in an ocean of acinar cells. Laguesse in 1893 named these mysterious cells the islands or islets of Langerhans. Oskar Minkowski in 1889 made the discovery that connected the pancreas and diabetes in his depancreatized dogs. Bliss, M., “The Discovery Of Insulin,” C. J. Pathol. 19:873-82 (1943). In 1901, while at Johns Hopkins University, Eugene Opie supplied a missing link by showing a pathological connection between diabetes and hyaline degeneration within the islet Langerhans. He described the presence of a hyaline staining substance currently referred to as islet amyloid and noted its association with diabetes mellitus. Opie, E. L., “The relation of diabetes mellitus to lesions of the pancreas: hyaline degeneration of the islands of Langerhans,” J. Exp. Med. 5:527-40 (1901). The amyloidal nature of this hyaline material was established by Ahronheim in 1943 and confirmed by alkaline Congo red staining by Ehrlick and Ratner in 1961. Ahronheim, J. H., “Nature of hyaline material in pancreatic islets in diabetes mellitus,” Am. J. Pathol. 19:873-82 (1943); Ehrlich J. C., Ratner I. M., “Amyloidosis of the islets of Langerhans. A restudy of islet hyaline in diabetic and non diabetic individuals,” Am. J. Pathol. 38:49-59 (1961).

In 1987, Cooper et al. were the first to report the discovery that this hyaline staining material consisted of a 37 amino acid monomer referred to as amylin. Cooper G. J. S., Willis A. C., “Purification and characterization of a peptide from amyloid-rich pancreas of type 2 diabetic patients,” Proc. Natl. Acad. Sci. USA 84:8628-32 (1987). Amyloid and type 2 diabetes are reviewed in Melvin R Hayden and Suresh C Tyagi, “‘A’ is for Amylin and Amyloid in Type 2 Diabetes Mellitus,” JOP. J. Pancreas (Online) 2(4):124-139 (2001). Thus, the major constituent of pancreatic islet amyloid is the 37-amino acid peptide hormone, amylin, which is normally secreted by β-cells within the pancreas (Cooper et al., Proc. Natl. Acad. Sci. U.S.A., 84:8628-8632 (1987); Cooper et al., Proc. Natl. Acad. Sci. U.S.A., 85:7763-7766 (1988); Cooper et al., Biochim. Biophys. Acta, 1014:247-258 (1989); Cooper, Endocr. Rev., 15:163-201 (1994); Cooper & Tse, Drugs & Aging, 9:202-212 (1996)). Amylin secretion is normally co-regulated and co-secreted with insulin production and is under the control of similar promoter and transcriptional elements. However, by mechanisms not fully understood, but likely resulting from over-secretion, amylin peptides interact to form fibrillar aggregates, known as islet amyloid (Cooper, Endocr. Rev., 15:163-201 (1994)). Other specific amylin molecules, such as those from monkeys and cats, contain amino acid sequences that also lead to the formation of amyloid fibrils (Cooper, Endocr. Rev., 15:163-201 (1994); Goldsbury et al., J. Struct. Biol., 119:17-27(1997); Goldsbury et al., J. Mol. Biol., 285:33-39(1999)). A detailed comparison of in vitro fibril formation by full-length human amylin (1-37) versus fragments of this peptide—human amylin (8-37) and human amylin (20-29)—has been made. It was reported that circular dichroism spectroscopy revealed that fibril formation was accompanied by a conformational change from random coil to β^{β}-sheet/α-helical structure. Fibril morphologies were visualized by electron microscopy and displayed formation of protofibrils of varying width and number. Goldsbury et al., “Amyloid Fibril Formation from Full-Length and Fragments of Amylin,” J. Structural Biol. 130(2-3): 352-362 (June 2000). See also Walsh et al., “Amyloid Beta-Protein Fibrillogenesis,” J. Biol. Chem. 274(36):25945-25952 (1999).

Islet amyloid is associated with a larger class of amyloid pathologies that are implicated in several diseases such as Alzheimer's Disease, immunoglobulin light chain amyloidosis, various organ and systemic amyloidoses, and the prion encephalopathies (Tjemberg et al., J. Biol Chem, 274:12619-12625 (1999); Sipe & Cohen, J Struct. Biol., 130:88-98 (2000); Collinge, Annu Rev Neurosci, 24:519-550 (2001); Jaikaran & Clark, Biochim. Biophys. Acta, 1537:179-203 (2001); Prusiner, N. Engl. J. Med., 344:1516-1526 (2001)). Alzheimer's disease is a neurodegenerative condition characterised by neuronal loss and the associated occurrence of extracellular senile plaques and neurofibrillary tangles (Lanza et al., Nature Biotechnology, 14:1107-1111 (1996); Yankner, Naure Medicine, 2:850-852 (1996); Selkoe, Nature, 399:A23-31 (1999)). The amyloid deposits are composed primarily of polymeric forms of β-amyloid peptide (Aβ) (Goldsbury et al., Trends Mol. Med., 7:582 (2001)). Prion diseases are also neurodegenerative conditions that are composed primarily of corrupted forms of a normal cellular host prion protein, PrPc (Collinge, Annual Rev. Neurosci., 24:519-550 (2001)). There is no known structural homology between the proteins that comprise these various amyloidoses, (Sipe & Cohen, J. Struct. Biol., 130:88-98 (2000)), but there are fundamental differences, particularly between islet amyloid and the amyloid structures seen in Alzheimer's disease and the prion encephalopathies (Tjemberg et al., J Biol Chem, 274:12619-12625 (1999); Goldsbury et al., J Struct Biol, 130:217-231 (2000); Baskakov et al., J. Biol. Chem., 276:19687-19690(2001); Collinge, Annual Rev. Neurosci., 24:519-550 (2001); Goldsbury et al., Trends Mol. Med., 7:582 (2001); Kallberg et al., J. Biol. Chem., 276:12945-12950 (2001); Yang et al., Amyloid, 8:10-19 (2001)); (Goldsbury et al., J. Struct. Biol., 119:17-27 (1997); Goldsbury et al., J. Mol. Biol., 285:33-39 (1999); Goldsbury et al., J. Struct. Biol., 130:352-362 (2000); Jaikaran & Clark, Biochim Biophys Acta, 1537:179-203 (2001)).

Circular dichroism spectroscopy has shown that islet amyloid fibril formation is accompanied by a conformational change from a random coil to β-sheet/α-helical structure (Goldsbury et al., J. Struct. Biol., 130:352-362 (2000)). In contrast, both the Alzheimer and prion amyloidoses comprise a distinct class of amyloid-forming proteins in which amyloid formation is accompanied by a reduction in α-helix content and an increase in β-sheet structure (Barrow et al., J. Mol. Biol., 225:1075-1093 (1992); Pan et al., Proc. Natl. Acad. Sci. U.S.A., 90:10962-10966 (1993)). In particular, Aβ and PrP harbor an α-helix in a polypeptide segment that should form a β-strand (Kallberg et al., J Biol Chem, 276:12945-12950 (2001)). In the PrPc this region occurs at helix 2, positions 179-191, while for the Alzheimer Aβ-peptide this discordance occurs at positions 16-23. When residues 14-23 are removed or changed to a nondiscordant sequence, Aβ fibrils are no longer formed (Kallberg et al., J. Biol Chem., 276:12945-12950 (2001)). The same inhibitory effect can be produced by incubation of Aβ with a pentapeptide corresponding to residues 16-20 (Tjernberg et al., J. Biol. Chem., 271:8545-8548 (1996)). Consequently, α-helix/β-strand discordant stretches are associated with this class of amyloid fibril formation, and in the cases of Aβ and PrPc, involve a transition from an α-helical structure to β-strand formation, see (Kallberg et al., J Biol Chem, 276:12945-12950 (2001)). These findings support the idea that distinct structure/function relationships exist between islet amyloid and other amyloid pathologies.

Previous research relating to Alzheimer's disease and the prion encephalopathies have focused on the polymerization properties of Aβ (Mazziotti & Perlmutter, Biochem. J., 332 (Pt 2):517-524 (1998); Bohrmann-et al., J. Biol. Chem. 274:15990-15995 (1999); Tjemberg et al., J. Biol. Chem., 274:12619-12625 (1999); Tjemberg et al., Chem. Biol., 6:53-62 (1999); Goldsbury et al., J Struct. Biol., 130:217-231 (2000); Jensen et al., Mol. Med., 6:291-302 (2000); Lannfelt & Nordstedt, J. Neural. Transm. Suppl., 59:155-161 (2000); Nunomura et al., J. Neuropathol. Exp. Neurol., 59:1011-1017 (2000); Chishti et al., J. Biol. Chem., 276:21562-21570 (2001); Yang et al., Amyloid, 8:10-19 (2001)), and the conversion of the normal cellular prion protein, PrPc, into the corresponding scrapie isoform, PrP^{Sc} (Hill et al., Proc. Natl. Acad. Sci. USA, 97:10248-10253 (2000); Kourie & Shorthouse, Am. J. Physiol. Cell. Physiol., 278:C1063-1087 (2000); Thellung et al., Int. J. Dev. Neurosci., 18:481-492 (2000); Baskakov et al., J. Biol. Chem., 276:19687-19690 (2001); Jackson & Collinge, Mol. Pathol., 54:393-399 (2001); Jansen et al., Biol. Chem., 382:683-691 (2001); Prusiner, N. Engl. J. Med., 344:1516-1526 (2001); Rudd et al., Biochemistry, 40:3759-3766 (2001); Tagliavini et al., Adv. Protein Chem., 57:171-201 (2001)) respectively. As these amyloidoses may be either associated with, or responsible for, the disease pathology, numerous studies have focused on strategies in which to obstruct amyloid formation in vivo. For Aβ this has led to the studies of certain peptide and non-peptide compounds in an effort to modulate fibril formation, as measured by various in vitro assays (Tjemberg et al., J. Biol. Chem., 271:8545-8548 (1996); Bohrmann et al., J. Biol. Chem., 274:15990-15995 (1999); Chyan et al., J. Biol. Chem., 274:21937-21942 (1999); Findeis & Molineaux, Methods Enzymol, 309:476-488 (1999); Findeis et al., Biochemistry, 38:6791-6800 (1999); Bohrmann et al., J. Struct. Biol., 130:232-246 (2000); Findeis, Biochim. Biophys. Acta., 1502:76-84 (2000); Kuner et al., J. Biol. Chem., 275:1673-1678 (2000); Forloni et al., FEBS Lett., 487:404-407 (2001); Poeggeler et al., Biochemistry, 40:14995-15001 (2001)).

Using electron microscopy, a thioflavin-T binding assay, and susceptibility to trypsin digestion, the classical antibiotics, tetracycline and doxycycline, reportedly appeared to modulate Aβ formation and defibrillate existing amyloid (Forloni et al., FEBS Lett., 487:404-407 (2001)). Another type of anthracycline, 4′iodo-4′-deoxydoxorubicin (IDOX) also reportedly inhibited formation of A β a myloid formation, as well as other amyloid forming proteins both in vitro and in vivo (Merlini et al., Proc. Natl. Acad. Sci. USA, 92:2959-2963 (1995)). The authors speculated that IDOX reduced Aβ amyloid formation and increased the solubility of existing plaques, thereby facilitating clearance by normal cell mechanisms (Merlini et al., Proc. Natl. Acad. Sci. USA, 92:2959-2963 (1995)). Szarek et al. (U.S. Patent Application 20010027186 (May 17, 2001) asserted the disruption of Aβ-amyloid by compounds containing phosphonate and carboxylate groups. See also U.S. Pat. No. 5,869,469 issued on Feb. 9, 1999 to Szarek, et al. for “Phosphonocarboxylate compounds for treating amyloidosis,” which is said to relate to methods for modulating amyloid deposition in a subject by administration of a compound comprising a phosphonate group and a carboxylate group, or a pharmaceutically acceptable salt or ester thereof. The patent asserts that in preferred embodiments, an interaction between an amyloidogenic protein and a basement membrane constituent is modulated.

Congo red, a compound used generally as an amyloid stain (Khurana et al., J. Biol. Chem., 276:22715-22721 (2001)), and various derivatives have also been asserted to inhibit Aβ amyloid neurotoxicity in cell cultures, possibly through stabilization of the Aβ pre-amyloid monomer (Lorenzo & Yankner, Proc. Natl. Acad. Sci. USA, 91:12243-12247 (1994); Findeis, Biochim. Biophys. Acta., 1502:76-84 (2000)). See also U.S. Pat. No. 5,276,059, issued on Jan. 4, 1994 to Caughey and Race for “Inhibition of diseases associated with amyloid formation.” The patent states that Congo Red may be used in a method of identifying a mammal having a condition associated with deposition of amyloidogenic protein in plaques and “administering to the mammal a pharmacologically effective amount of or a pharmaceutically acceptable salt or derivative thereof in an amount sufficient to interfere with amyloidogenic protein formation or to destabilize amyloidogenic protein structures already formed in the mammal.” The patent further states that the method contemplates the treatment of a large number of such amyloidogenic diseases, and the “preferred form of the invention” is said to be the treatment, prevention and/or inhibition “conditions associating with plaques occurring in a tissue of the central nervous system.” In another form, the method is said to be useful against a disease of the internal organs related to amyloid plaque formation, including plaques in the pancreas and the “treatment of Adult type II diabetes where the plaques occur in the pancreas.” Other strategies for the prion encephalopathies have also been reported recently (Aguzzi et al., Nat. Rev. Neurosci., 2:745-749 (2001)). Prions are composed exclusively of SC a misfolded prion protein isoform, PrP^{Sc}, resulting from a major conformational change of PrP^{c}, a normal host encoded glycolipid-anchored protein (Collinge, Annu. Rev. Neurosci., 24:519-550 (2001)). Reported findings with acridine and phenothiazine derivatives led the authors to suggest various compounds as intermediate candidates for the treatment of Creutzfeldt-Jakob disease and other for prion diseases (Korth et al., Proc. Natl. Acad. Sci. USA, 98:9836-9841 (2001)). A range of tricyclic compounds were tested, and chlorpromazine and quinacrine were also reportedly effective in reversal of disease-forming prion plaques in scrapie infected mouse cell cultures (Korth et al., Proc. Natl. Acad. Sci. USA, 98:9836-9841 (2001)). The authors noted that an aliphatic side chain on the central tricyclic ring was necessary for maximal inhibition of prion plaque formation. Id. In another study, tetracycline was reported to: (i) bind and inhibit the assembly of amyloid fibrils generated by synthetic peptides corresponding to residues 106-126 and 82-146 of human PrPc; (ii) remove the protease resistance of PrP peptide aggregates and PrP^{Sc} extracted from brain tissue of patients with Creutzfeldt-Jakob disease; (iii) prevent neuronal death and astrocyte proliferation induced by PrP peptides in vitro. NMR spectroscopy also reportedly revealed several space interactions between aromatic protons of tetracycline and side-chain protons of Ala(117-119), Val(121-122) and Leu(125) of PrP 106-126 (Tagliavini et al., J. Mol. Biol., 300:1309-1322 (2000)).

The role of islet amyloid in the pathogenesis of type 2 diabetes mellitus has been debated and is still unclear (Cooper, Endocr. Rev., 15:163-201 (1994); Cooper & Tse, Drugs & Aging, 9:202-212 (1996); Cooper, Handbook of Physiology. Section 7: The Endocrine system. Volume II: The endocrine pancreas and regulation of metabolism (2001)). However, it is proposed herein that chronic deposition of islet amyloid promotes β-cell loss and is a significant factor contributing to β-cell dysfunction in late stage type 2 diabetes mellitus. This is supported by several emerging lines of evidence. First, human amylin forms islet amyloid in most patients with type 2 diabetes (Hoppener et al., N. Engl. J. Med., 343:411-419 (2000); Jaikaran & Clark, Biochim Biophys Acta, 1537:179-203 (2001)). Second, human amylin, but not non-fibril-forming variants, causes death of pancreatic islet β-cells in culture (Lorenzo & Yankner, Proc. Natl. Acad. Sci. USA, 91:12243-12247 (1994); Bai et al., Biochem. J, 343 Pt 1:53-61 (1999)). Third, site-specific expression of human amylin in the pancreas of transgenic mice reproduces diabetes-like syndromes through β-cell loss (Verchere et al., Hormone & Metabolic Research, 29:311-316 (1997); Soeller et al., Diabetes, 47:743-750 (1998); Hoppener et al., Diabetologia, 42:427-434 (1999)).

Others have attempted different avenues of treatment of amyloid-related disease than those described and claimed herein. U.S. Pat. No. 5,854,204, issued to Findeis, et al. for “αβ peptides that modulate P-amyloid aggregation” proposes the use of an amyloidogenic protein, or peptide fragment thereof, coupled directly or indirectly to at least one modifying group such that the compound modulates the aggregation of natural amyloid proteins or peptides when contacted with the natural amyloidogenic proteins or peptides. The patent states that the amyloidogenic protein or fragment can be transthyretin (TTR), prion protein (PrP), islet amyloid polypeptide (IAPP), atrial natriuretic factor (ANF), kappa light chain, lambda light chain, amyloid A, procalcitonin, cystatin C, β2 microglobulin, ApoA-I, gelsolin, calcitonin, fibrinogen or lysozyme.

U.S. Pat. No. 5,859,001 issued on Jan. 12, 1999 to Simpkins, et al. for “Neuroprotective effects of polycyclic phenolic compounds” is said to relate to the use of non-estrogen compounds having a terminal phenol group in a four-ring cyclopentanophenanthrene compound structure for conferring neuroprotection to cells and for the treatment of neurodegenerative diseases. See also U.S. Pat. No. 6,197,833 issued on Mar. 6, 2001 to Simpkins, et al. for “Neuroprotective effects of polycyclic phenolic compounds.”

U.S. Pat. No. 6,221,667 issued on Apr. 24, 2001 to Reiner, et al. for “Method and composition for modulating amyloidosis” is said to relate to methods and compositions asserted to be useful in the treatment of amyloidosis and conditions and diseases associated therewith, such as Alzheimer's Disease, by the administration of agents that modulate amyloidosis precursor protein catabolism and amyloid deposition for use in inhibiting amyloidosis in disorders in which amyloid deposition occurs. The methods are said to be based on modulating catabolism of amyloidosis precursor protein in amyloidosis precursor protein-containing cells through the use of a mobile ionophore, such as carbonyl cyanide p-(trifluoromethoxy)phenylhydrazone.

U.S. Pat. No. 6,277,826 issued on Aug. 21, 2001 to Findeis, et al. for “Modulators of β-amyloid peptide aggregation comprising β-amino acids” is said to relate to peptides comprised entirely of D-amino acids that modulate natural β amyloid peptide aggregation. The peptides are said to be preferably based on a β amyloid peptide, and preferably comprise 3-5 D-amino acid residues and include at least two D-amino acid residues independently selected from D-leucine, D-phenylalanine and D-valine. In a particularly preferred embodiment, the patent states that the peptide is a retro-inverso isomer of a β amyloid peptide, and that in certain embodiments the peptide is modified at the amino-terminus, the carboxy-terminus, or both. Preferred amino-terminal modifying groups are said to include cyclic, heterocyclic, polycyclic and branched alkyl groups, and preferred carboxy-terminal modifying groups are said to include an amide group, an alkyl amide group, an aryl amide group or a hydroxy group. See also U.S. Pat. No. 6,303,567 issued on Oct. 16, 2001 to Findeis, et al. for “Modulators of β-amyloid peptide aggregation comprising D-amino acids”

At the present time, no compounds have been demonstrated that interfere with islet amyloid formation or disrupt existing islet amyloid. Congo red reportedly inhibited human amylin toxicity in pancreatic cells in vitro, but not amylin fibril formation (Lorenzo & Yankner, Proc. Natl. Acad. Sci. USA, 91:12243-12247 (1994)). In contrast, Congo red reportedly inhibits Aβ amyloid neurotoxicity by inhibiting fibril formation or by binding to preformed Aβ fibrils. Id.

There exists a need for additional methods for blocking amyloid protein production and for blocking toxicity associated with the transition from soluble amylin to insoluble amylin and to block the formation of protofibrils. There is also a need for blocking amyloid β-peptide toxicity in neurons, inhibiting the production of amyloid beta peptide, and blocking the production of various other cytotoxic amyloid proteins that result in disease conditions.

### SUMMARY OF THE INVENTION

The present invention provides methods of blocking amyloid protein toxicity in cells using one or more of defined classes of polycyclic compounds. Also provided are methods of decreasing amyloid protein production in cells. The compounds and methods of the invention can be used to prevent and treat a diverse class of disease conditions, known as amyloidoses, which are all the result of amyloid protein deposits.

In accordance with another aspect of the invention, there are provided methods of identifying compounds that can block toxicity normally associated with amyloid resulting from the transition from soluble amylin to insoluble amylin and the formation of protofibrils.

There are also provided methods of identifying active, cytotoxic conformers of various amyloidoses peptides and preventing their formation. Also provided are methods for screening compounds useful in the methods of the invention, including compounds that are effective in disrupting the transition from a soluble to an insoluble form of amylin, inhibiting aggregation of amyloid pre-fibrils and fibrils, and inhibiting transition-induced toxicity of amyloid β-fibril and β-sheet formation.

The present invention further relates to the treatment of type 2 diabetes mellitus and to medicaments for use therein. Thus, in one aspect, the present invention consists of a method of treating type 2 diabetes mellitus in a subject, preferably a human or other mammalian subject, or other suitable individual having a need which comprises disruption of islet amyloid from within, or extracellular to, islet β-cells by administration of a suitable polycyclic compound of the invention. In a further aspect, the present invention consists of a method of treating type 2 diabetes mellitus in a subject, preferably a human or other mammalian subject, or other suitable individual having a need which comprises disruption of amylin protofibril formation from within, or extracellular to, islet β-cells by administration of a suitable polycyclic compound of the invention. In yet a further aspect, the present invention consists of a method of treating a subject, preferably a human or other mammalian subject, or other suitable individual having a need through protection of islet β-cells of said patient against death, through disruption of human islet amyloid or formation of amylin protofibrils from within, or extracellular to, said β-cells. In yet a further aspect, the present invention consists of a method of treating a subject, preferably a human or other mammalian subject, or other suitable individual having a need, including but not limited to subjects with or at risk for type 2 diabetes mellitus that results in improvement of, or reduction in, deterioration of islet β-cell function following treatment with polycyclic compounds described and claimed herein. In yet a further aspect, the present invention consists of a method of treating a subject, preferably a human or other mammalian subject, or other suitable individual having a need, including but not limited to subjects with or at risk for type 2 diabetes mellitus which comprises disruption of islet amyloid from within, or extracellular to, said islet β-cells, and which aids clearance of islet amyloid. In yet a further aspect, the present invention consists of a method of treating a subject, preferably a human or other mammalian subject, or other suitable individual having a need, including but not limited to subjects with or at risk for type 2 diabetes mellitus which comprises disruption of human islet amyloid and which aids immune recognition and clearance of islet amyloid. In yet a further aspect, the present invention consists of a method of treating a subject, preferably a human or other mammalian subject, or other suitable individual having a need, including but not limited to subjects with or at risk for type 2 diabetes mellitus, which comprises the co-treatment of said subject with a polycyclic compound of the invention in combination with an adjunctive treatment, such as immunotherapy, which promotes in vivo clearance of islet amyloid or islet amyloid precursors or islet amyloid protofibrils. In yet a further aspect, the present invention consists of a method of treating a subject, preferably a human or other mammalian subject, or other suitable individual having a need, including but not limited to subjects with or at risk for type 2 diabetes mellitus that results in improvement of, or reduction in deterioration of islet β-cell function following co-treatment with polycyclic compounds of the invention in combination with other adjunctive therapies, such as immunotherapy, that stimulate in vivo clearance mechanisms of islet amyloid, islet amyloid precursors, or islet amyloid protofibrils. In yet a further aspect, the present invention consists of a method of treating a subject, preferably a human or other mammalian subject, or other suitable individual having a need, including but not limited to subjects with or at risk for type 2 diabetes mellitus which comprises or includes co-treatment of the said patient with a polycyclic compound of the invention and an adjunctive treatment, such as immunotherapy, which together cause disruption of pre-formed human islet amyloid and/or inhibits the formation of islet amyloid from amylin within said P-cells.

In yet a further aspect, the present invention consists of a method for measurement of islet amyloid disruption in vitro by polycyclic compounds using thioflavin-T enhanced fluorescence, radioactive amyloid precipitiation assays, electron microscopy and measurement of islet amyloid cytotoxicity in cultured islet β-cells, and circular dichroism.

In yet a further aspect, the present invention relates to a method of preventing an amyloid-associated disease comprising preventing protofibril formation and/or reduction of existing protofibril deposits.

In yet a further aspect, the present invention consists of a method of screening polycyclic compounds as potential drugs for islet amyloid disruption in vitro by using thioflavin-T enhanced fluorescence, radioactive amyloid precipitiation assays, electron microscopy and measurement of islet amyloid cytotoxicity in cultured islet β-cells, and circular dichroism.

In yet a further aspect the present invention consists of the use of a polycyclic compound or polycyclic compounds of the invention in the manufacture of a pharmaceutical composition comprising or including the polycyclic compound(s) and a suitable pharmaceutical carrier therefor and which composition is useful in treating a subject, preferably a human or other mammalian subject, or other suitable individual having a need, including but not limited to subjects suffering from type 2 diabetes mellitus or at risk for developing type 2 diabetes by one or more of the following: (i) disruption of pre-formed human islet amyloid; (ii) inhibition of the formation of subsequent islet amyloid from amylin; (iii) improvement of islet β-cell function; and/or, (iv) reduction in the deterioration of islet β-cell function.

The present invention includes and is not limited to methods of treatment as previously and/or herein described on all mammalian species with type 2 diabetes mellitus or who are otherwise at risk for developing type 2 diabetes or pancreatic islet amyloid or islet β-cell dysfunction.

Applicants' invention is directed to methods for the preparation and/or manufacture of a medicament(s) using the one or more of the compounds disclosed, identified, and/or claimed herein for the treatment of one or more of the disorders, diseases, conditions and/or purposes referred to herein.

### BRIEF DESCRIPTION OF THE DRAWINGS

FIG. 1 shows the effect of tetracycline and congo red on enhancement of thioflavin-T fluorescence by human amylin.

FIG. 2 shows the effect of chlorpromazine on enhancement of thioflavin-T fluorescence by human amylin.

FIG. 3 shows the effect of selected polycylic compounds on enhancement of thioflavin-T fluorescence by human amylin.

FIG. 4 shows electron micrographs of human amylin fibrils in the presence and absence of tetracycline.

FIG. 5 shows electron micrographs of human amylin fibrils in the presence and absence of quinacrine.

FIG. 6 shows electron micrographs of human amylin fibrils in the presence and absence of selected polycyclic compounds.

FIG. 7 shows the effect of tetracycline on amylin fibril formation by radiolabelled precipitation.

FIG. 8 shows the effect of selected polycyclics on amylin fibril formation by radiolabelled precipitation.

FIG. 9 shows the effect of Congo red on amylin fibril formation by circular dichroism.

FIG. 10 shows the p rotective effect of Congo red against amylin fibril-mediated toxicity in RINm5F cells.

### DETAILED DESCRIPTION OF THE INVENTION

Applicants have discovered that certain polycyclic compounds can be used to disrupt islet amyloid formation and for the treatment of type 2 diabetes mellitus. The strategy does not depend upon the blockade of amylin action mediated via a receptor-mediated mechanism. Instead, without being bound by any specific mechanism on the cytotoxic component of islet amyloid, the strategy targets the disruption of islet amyloid formation. In a clinical setting, modulation of islet amyloid, in vivo, in combination with endogenous clearance mechanisms, has the potential to improve β-cell function or prevent reduction in further β-cell dysfunction and loss.

The application discloses that the cytotoxic effect results from the transition from soluble to insoluble amylin and the formation of β-strands leading to the common β-pleated sheet regardless of actual fibril formation. Further, disruption of this transition can protect β-islet cells from cell death.

Islet amyloid is herein defined as comprising human amylin as either insoluble islet amyloid or as soluble amyloid precursors formed through the aggregation of monomeric human amylin, or as any form of human amylin that is cytotoxic to islet β-cells. As referred to in the text, human amylin fibrils are also defined herein as components of islet amyloid.

Disruption of islet amyloid by polycyclic compounds is defined herein as the whole or partial conversion of insoluble human islet amyloid to soluble precursors, and/or the reduction in the rate of, or prevention of, the formation of islet amyloid from human amylin. Included in this definition are interactions of polycyclic compounds with islet amyloid that result in changes to the cytotoxic properties of islet amyloid to islet β-cells. This application recognises a prospect of effectively using non-peptide molecules to (i) slow the rate of, or inhibit formation of human islet amyloid, and/or (ii) disrupt existing forms of human islet amyloid. We believe a method that uses non-peptide polycyclic compounds can achieve both (i) and (ii), either as an exclusive treatment or in combination with other therapeutic treatments.

The present invention recognizes that polycyclic compounds have application by simple dosage regimes in the treatment of common ailments (Drisko, J. Clin. Periodontol., 25:947-952 (1998); Klein & Cunha, Med. Clin. North Am., 85:125-132 (2001)). Particularly appropriate are those compositions administered into humans by a route of convenience such as orally or parenterally which lends these compounds to chronic application in patients at risk to or already subject to type 2 diabetes mellitus.

As used herein, reference to “a polycyclic compound” can also include combinations of appropriate polycyclic compounds. Polycyclic compounds that disrupt islet amyloid, include compounds such as quinacrine (an anti-malarial compound), chlorpromazine (an anti-psychotic compound), and tetracycline (an antibiotic compound). Other structures (e.g., acridine, phenothiazines, anthracyclines, and combinations of fused rings and biphenyl structures) possessing a core structure of tetracycline, quinacrine, chlorpromazine and Congo red are also polycyclic compounds of the present invention.

In accordance with the present invention, there are provided methods of blocking toxicity normally associated with amyloid resulting from the transition from soluble amylin to insoluble amylin and the formation of protofibrils in cells, said methods comprising contacting said cells with an effective amount of at least one polycyclic compound selected from the group of polycyclic compounds contained herein. Specifically, the group of polycyclic compounds includes anthracene, phenalene, phenanthrene, quinacrine, acridine acridine orange, neutral red, chloropromazine, methylene blue, phenothiazine, pyrene, chrysene, benz[a]anthracene, benz[m]anthracene, benz[c]phenanthrene, and tetracene. These molecules share a common structure of a multiple ring structure. Molecules having a similar multiple ring structure may also be used.

Invention methods can optionally be effected using pharmaceutically acceptable salts of the above-described compounds. Such salts are generally prepared by reacting the compounds with a suitable organic or inorganic acid or base. Representative organic salts include methanesulfonate, acetate, oxalate, adipate, alginate, aspartate, valerate, oleate, laurate, borate, benzoate, lactate, phosphate, toluenesulfonate (tosylate), citrate, malate, maleate, fumarate, succinate, tartrate, napsylate, methanesulfonate, 2-naphthalenesulfonate, nicotinate, benzenesulfonate, butyrate, camphorate, camphorsulfonate, cyclopentanepropionate, digluconate, dodecylsulfate, glucoheptanoate, glycerophosphate, heptanoate, hexanoate, undecanoate, 2-hydroxyethanesulfonate, ethanesulfonate, and the like. Representative inorganic salts can be formed from inorganic acids such as sulfate, bisulfate, hemisulfate, hydrochloride, chlorate, perchlorate, hydrobromide, hydroiodide, and the like. Examples of a base salt include ammonium salts; alkali metal salts such as sodium salts, potassium salts, and the like; alkaline earth metal salts such as calcium salts, magnesium salts, and the like; salts with organic bases such as dicyclohexylamine salts, N-methyl-D-glucamine, phenylethylamine, and the like; and salts with amino acids such as arginine, lysine, and the like. Such salts can readily be prepared employing methods well known in the art.

As used herein, the phrase “blocking toxicity normally associated with amyloid” refers to preventing or inhibiting the harmful and lethal effects on cells and tissues caused by the transition from soluble amylin to insoluble amylin and the formation of protofibrils.

As used herein, the term “β-conformer” refers to non-monomeric amylin which possesses some degree of β-sheet structure.

As used herein, the term “protofibril” refers to individual protofibrils and higher order protofibril structures including oligomeric forms of human amylin or other mammalian amylin variants which show some amount of β-conformer and are not monomeric.

While not bound by any one mechanism, it is recognized that compositions that can interfere with the transition from soluble amylin to insoluble amylin and the formation of protofibrils are able to block toxicity normally associated with amyloid. These compounds include polycyclic compounds such as those described herein.

In addition, compositions that prevent or inhibit the production of amyloid peptide, and compounds that prevent or inhibit the formation of amyloid fibrils are able to block toxicity normally associated with amyloid.

Conventional wisdom has been that at excessive levels, amyloid proteins have a cytotoxic effect on cells, resulting in cell death. Applicants discovered that conformational changes (e.g. from soluble amylin to insoluble amylin, β-conformers and/or protofibril formation desired targets for therapy.

Further, it was previously believed that aggregation of amyloid proteins into amyloid fibrils is likely required for its cytotoxic effect, however, the biochemical mechanisms underlying amyloid toxicity were not well understood. Applicants demonstrate that aggregation is not necessary to effect cytotoxicity; however, inhibiting the aggregation of amyloid proteins is another means of reducing cytotoxicity.

As used herein, “amyloid toxicity” refers to any deleterious effect of amyloid protein on cells, especially said conformational transition or pre-fibril formation resulting in a deleterious effect.

As used herein, the phrase “contacting” refers to providing compounds to cells or cellular targets. Contacting may take place in solid, liquid or gaseous phase, and refers to events that take place extracellularly and intracellularly. Those of skill in the art will recognize that providing compounds to cells in vivo may be accomplished by n uinerous-modes of administration, including oral, sublingual, intravenous, subcutaneous, transcutaneous, intramuscular, intracutaneous, intrathecal, epidural, intraoccular, intracranial, inhalation, rectal, vaginal, and the like.

As employed herein, the phrase “effective amount,” when used in reference to invention methods employing polycyclic compounds, refers to a dose of compound sufficient to provide concentrations high enough to effect the desired result. The specific effective amount for any one compound will depend upon a variety of factors including the type of cell, the timing of the administration, the severity of the disorder, the activity of the specific compound used, the route of administration, the rate of clearance of the specific compound, the duration of exposure of the cells to the compound, the drugs used in combination or coincident with the specific compound, and the like.

In one embodiment of the present invention, there are provided methods of blocking amyloid toxicity by interrupting the transition from soluble amylin to insoluble amylin and the formation of protofibrils wherein the amyloid toxicity is amyloid beta peptide toxicity. As employed herein, “amyloid beta peptide” (Aβ) refers to proteins of about 40 to 43 amino acid residues. Amyloid beta peptide is predominantly in a 40 amino acid form, i.e., amyloid beta 1-40, however amyloid beta 1-42 and amyloid beta 1-43 are also associated with amyloid fibrils and deposits. The proteins are derived by proteolytic cleavage from their much larger precursor which is known as amyloid beta precursor protein (AβPP). AβPP, a member of a family of amyloid precursor-like proteins, exists in three principal isoforms of 695, 751 and 770 amino acid residues, respectively, each of which contain the amino acid sequence of an amyloid beta peptide. AβPP is synthesized in the rough endoplasmic reticulum, and delivered to the cell surface as an integral membrane protein. AβPP is present in the dendrites, cell bodies and axons of neurons, although its normal neuronal functions are not yet understood. Some of the AβPP in the plasmalemma is internalized into the cell where it is enzymatically processed to an amyloid beta peptide.

AβPP undergoes proteolytic cleavage by several secretases to give rise to various forms of amyloid beta peptide. One type of secretase, gamma-secretase, cleaves AβPP in the carboxy-terminal region of the precursor to generate a single copy of an amyloid beta peptide from each precursor molecule. Another type of secretase, alpha-secretase, cleaves the precursor within the amyloid beta sequence and therefore, cleavage by this secretase does not produce an amyloid beta peptide.

Amyloid beta peptides are the major constituent of the senile plaques found in the central nervous system of patients with Alzheimer's disease. Senile (or neuritic plaques), comprising extracellular deposits of amyloid beta protein, dystrophic axons, and processes of astrocytes and microglia, are distributed throughout the neuropil and in the walls of the cerebral blood vessels.

In accordance with another embodiment of the present invention, there are provided methods of blocking amyloid toxicity wherein the amyloid toxicity is prion protein toxicity. As employed herein, “prion protein” refers to products of the human prion gene (termed PRNP) located on the short arm of chromosome 20 and which has an open reading frame consisting of a single exon encoding 254 residues. The normal prion gene product, prion protein (PrP) is a constitutively expressed cell-surface glycoprotein that is bound to the plasmalemma by a glycolipid anchor. The highest levels of PrP messenger RNA are found in neurons of the central nervous system, but the function of the protein is unknown. The normal cellular prion protein and the infectious prion protein do not differ in amino acid sequence, but, similar to amyloid proteins, the normal and infectious proteins have different three-dimensional configurations. Normal prion protein is rich in α-helices, having four putative domains, and little beta-pleated sheet configuration. In contrast, the infectious protein has increased beta-pleated sheet configuration. The normal and infectious proteins also have different patterns of glycosylation (see Pathology, 3.sup.rd ed.(1999) Rubin and Farber, eds., Lippincott-Raven, pp. 1492-1496).

In accordance with still another embodiment of the present invention, there are provided methods of blocking amyloid toxicity, wherein the amyloid toxicity is amylin toxicity. As used herein, “amylin” refers to a polypeptide which is secreted along with insulin by the β-cells in the islets of Langerhans. Amylin is a 37-residue. C-terminally amidated peptide having a disulfide bridge between the cysteines at residues 2 and 7, and various segments within the sequence are sufficient to form β-sheet-containing amyloid fibrils (e.g., Nilsson and Raleigh, J. Mol. Biol. 294:1375-85; Rhoades et al., Biochim Biophys Acta 1476:230-8 (2000); and Tenidis et al., J. Mol. Biol. 295:1055-1071 (2000)). Pancreatic amyloid is found in more than 95% of type 2 diabetes patients and is formed by the aggregation of amylin.

In accordance with yet another embodiment of the present invention, there are provided methods of blocking amyloid toxicity resulting from the transition from soluble amylin to insoluble amylin and the formation of protofibrils, wherein the amyloid toxicity is amyloid A protein toxicity. As used herein, “amyloid A protein” refers to a polypeptide of about 76 amino acids that is derived from a larger precursor lipoprotein synthesized primarily in the liver, and called serum amyloid A (SAA). Following stimulation of SAA synthesis, SAA is denatured, thereby releasing into the circulation a subunit termed apoSAA, which is internalized by reticuloendothelial cells. Upon release from the reticuloendothelial cells into a fibrillogenic environment containing glycosaminoglycans, serum amyloid P, laminin, collagen IV and Apo E, amyloid fibrils may form, allowing the formation of amyloid deposits (see Pathology, 3.sup.rd ed. supra, pp. 1228-1²29).

In accordance with still another embodiment of the present invention, there are provided methods of blocking amyloid toxicity, wherein the amyloid toxicity is transthyretin toxicity. As used herein, “transthyretin” (TTR) refers to a mutated form of a protein that is secreted by the liver into the plasma, where its normal function is to serve as a carrier of thyroid hormones and as a retinal binding protein. At least 60 mutant forms of the protein have been described, each giving rise to a clinical variant of a familial amyloidotic polyneuropathy (FAP). The most common variant of FAP is due to transthyretin, where there is an amino acid substitution at residue 30 of methionine for valine. The sequence modification lowers the stability of the tetrameric TTR, allowing the formation of a monomeric intermediate with an altered conformation (see Pathology, 3.sup.rd ed. supra, pp. 1225, 1228).

In accordance with yet another embodiment of the present invention, there are provided methods of blocking amyloid toxicity, wherein the amyloid toxicity is AL amyloid toxicity. As used herein, “AL amyloid” refers to a protein that consists of the variable region of immunoglbulin light chains and can be derived from either the kappa or lambda moieties. Excess production of immunoglobulins results in their secretion into the circulatory system which provides a fibrillogenic environment due to the presence of glycosaminoglycans, serum amyloid P, laminin, collagen IV and ApoE. Amyloid fibrils that form are then processed proteolytically in various types of cells, including macrophages, Kupffer cells and endothelial cells, resulting in the formation of amyloid deposits (see Pathology, 3.sup.rd ed. supra, pp. 1226-1227).

In accordance with still another embodiment of the present invention, there are provided methods for decreasing amyloid protein production in cells, said method comprising contacting said cells with an effective amount of at least the compounds described herein, or enantiomers, diasteriomeric isomers or mixtures of any two or more thereof, or pharmaceutically acceptable salts thereof.

Decreasing amyloid protein production can block or prevent the cytotoxic effects on cells of excessive levels of amyloid protein, and block or prevent the formation of amyloid plaques, such as those associated with various amyloid-related diseases. Various means of decreasing amyloid protein production are contemplated including reducing or preventing the production of an amyloid precursor protein, reducing or preventing the proteolytic cleavage that generates amyloid protein, reducing or preventing post-translational modification of amyloid protein, reducing or preventing internalization of amyloid precursor protein by increasing membrane stabilization, and the like.

In preferred embodiments of the invention, amyloid protein production is blocked or prevented by decreasing amyloid beta peptide, amyloid prion protein, islet amyloid protein (amylin), amyloid A protein, transthyretin or AL amyloid.

In accordance with yet another embodiment of the invention, there are provided methods of inhibiting nerve cell death, said methods comprising contacting the nerve cells with an effective amount of at least one compound described or identified herein.

As used herein, “nerve cell death” refers to a reduction in nerve cell number or to a loss of nerve cell function. Nerve cell death can occur through activation or acceleration of an apoptotic pathway, i.e., programmed cell death, or through a necrotic cell death which does not involve activation of an endogenous cell death program. Necrotic cell deaths often result from acute traumatic injury and typically involve rapid lysis of cellular membranes. Inhibiting nerve cell death can reduce the loss of nerve cells or the loss of nerve cell function that is associated with both types of nerve cell death.

In accordance with still another embodiment of the present invention, methods are provided for treating a disease condition in a subject in need thereof, said method comprising administering to the subject a therapeutically effective amount of a compound described or identified herein.

As used herein, “treating” refers to inhibiting or arresting the development of a disease, disorder or condition and/or causing the reduction, remission, or regression of a disease, disorder or condition. Those of skill in the art will understand that various methodologies and assays may be used to assess the development of a disease, disorder or condition, and similarly, various methodologies and assays may be used to assess the reduction, remission or regression of a disease, disorder or condition.

Essentially, any disease that is etiologically linked to the formation and/or deposition of amyloid is contemplated for treatment according to the present invention. As used herein, “disease condition” refers to a disorder such as Alzheimer's disease, systemic senile amyloidosis, prion disease, scrapie, bovine spongiform encephalopathy, Creutzfeldt-Jakob disease, Gerstmann-Straussler-Scheinker syndrome, type 2 diabetes (or any diabetic or other condition characterized by, or that carries a risk for the development or increase in the amount of, islet amyloid, including insulinoma), amyloid A amyloidosis, AL amyloidosis, familial amyloid polyneuropathy (Portuguese, Japanese and Swedish types), familial transthyretin amyloidosis, familial Mediterranean Fever, familial amyloid nephropathy with urticaria and deafness (Muckle-Wells syndrome), hereditary non-neuropathic systemic amyloidosis (familial amyloid polyneuropathy III), familial amyloidosis of Finnish type, familial amyloid cardiomyopathy (Danish type), isolated cardiac amyloid, isolated atrial amyloidosis, idiopathic (primary) amyloidosis, myeloma or macroglobulinemia-associated amyloidosis, primary localized cutaneous nodular amyloidosis associated with Sjogren's syndrome, reactive (secondary) amyloidosis, hereditary cerebral hemorrhage with amyloidosis of Icelandic type, amyloidosis associated with long term hemodialysis, fibrinogen-associated hereditary renal amyloidosis, amyloidosis associated with medullary carcinoma of the thyroid, lysozyme-associated hereditary systemic amyloidosis, and the like.

Amyloid deposits are found in subjects diagnosed with Alzheimer's disease, a neurodegenerative disease characterized by atrophy of nerve cells in the cerebral cortex, subcortical areas, and hippocampus and the presence of plaques, dystrophic neurites and neurofibrillary tangles. In Alzheimer's disease, dystrophic or aberrant neurite growth, synapse loss, and neurofibrillary tangle formation are strong correlates of disease severity. Dystrophic neurons characteristically contain abundant electrodense multilaminar bodies in the cytoplasm of the neurites and have disruption of synaptic junctions. The dystrophic neurons surround deposits of amyloid, thereby forming the senile plaques located throughout the brain neuropil as well as in the walls of cerebral blood vessels. Invention methods for treating Alzheimer's disease can reduce or block the atrophy of nerve cells, reduce or block the formation of senile plaques or neurofibrillary tangles, and the like, such that the development of the disease is slowed or arrested.

Amyloid deposits are also found in the islets of Langerhans in patients diagnosed with type 2 diabetes. The deposits contain an amyloid protein that is derived from a larger precursor called amylin which, in normal animals, has a hormonal role. Amylin is produced by the beta cells of the islets and has a profound effect on glucose uptake by the liver and striated muscle cells. In transgenic mice having a transgene for human amylin and which are fed a high fat diet, overproduction of amylin leads to islet amyloid deposition (see Pathology, 3.sup.rd ed. (1999) supra, p. 1226). Invention methods for treating amyloid deposits in the islets of Langerhans in patients having type 2 diabetes can reduce or prevent the formation of amyloid protein, reduce or prevent the deposition of amyloid protein into amyloid deposits, and the like.

Yet another disease where amyloid deposits are noted is prion disease, one type of spongiform encephalopathy. Prion diseases are neurodegenerative conditions characterized clinically by progressive ataxia and dementia, and pathologically by vacuolization of spongiform brain tissue. Amyloid deposits are associated with at least one prion disease known as kuru. In kuru, about 70% of prion protein accumulates extracellulary to form plaques, in contrast to normal prion protein which is a constitutively expressed cell-surface glycoprotein (see Pathology, 3.sup.rd ed. supra, pp. 1492-1496). Invention methods for treating prion disease can reduce or prevent the production of amyloid protein, reduce or prevent the deposition of amyloid plaques, and the like.

Still another disease where amyloid deposits are noted is amyloid A amyloidosis. Amyloid A amyloidoses refer to amyloidoses from seemingly unrelated disorders such as chronic inflammatory disorders, neoplastic disorders, and hereditary disorders. The deposition of amyloid protein is secondary to the underlying disease condition. The precursor molecule is serum amyloid A (SAA), an acute phase reactant, which can be used as a surrogate marker of inflammation in many diseases. Invention methods for treating amyloid A amyloidosis can reduce or prevent the production of amyloid protein, reduce or prevent the production of the precursor to amyloid protein, prevent or reduce any one of several steps necessary to generate an active amyloid protein, reduce or prevent the deposition of amyloid plaques, and the like.

Yet another disease where amyloid deposits are noted is familial transthyretin amyloidosis which is the most common form of Familial Amyloidotic Polyneuropathy (FAP). The human amyloid disorders, familial amyloid polyneuropathy, familial amyloid cardiomyopathy and senile systemic amyloidosis, are caused by insoluble transthyretin (TTR) fibrils, which deposit in the peripheral nerves and heart tissue. Transthyretin is a homotetrameric plasma protein implicated in the transport of thyroxine and retinol. The most common amyloidogenic TTR variant is V30M-TTR, while L55P-TTR is the variant associated with the most aggressive form of FAP. Invention methods for treating amyloidoses caused by transthyretin can reduce or prevent the production of amyloid protein, reduce or prevent the production of the precursor to amyloid protein, prevent or reduce any one of several steps necessary to generate an active amyloid protein, reduce or prevent the deposition of amyloid plaques, and the like.

A further disease where amyloid deposits are noted is AL amyloidosis. AL amyloidosis is a class of diseases related to a primary disorder of immunoglobulin production which includes primary amyloidosis, plasma cell dyscrasia, immunoblastic lymphoma, multiple myeloma, and the like. Primary systemic AL (amyloid light-chain) amyloidosis is a plasma cell disorder in which depositions of amyloid light-chain protein cause progressive organ failure. The prognosis of primary amyloidosis is generally poor, with a median survival of 1-2 years. The precursor protein is an immunoglobulin light chain in both localized and systemic AL-amyloidosis which shows the same pattern of fragmentation and changes of primary structure. Invention methods for treating amyloidoses caused by AL amyloid proteins can reduce or prevent the production of amyloid protein, reduce or prevent the production of the precursor to amyloid protein, prevent or reduce any one of several steps necessary to generate an active amyloid protein, reduce or prevent the deposition of amyloid plaques, and the like.

As used herein, “administering” refers to providing a therapeutically effective amount of a compound to a subject, using oral, sublingual, intravenous, subcutaneous, transcutaneous, intramuscular, intracutaneous, intrathecal, epidural, intraoccular, intracranial, inhalation, rectal, vaginal, and the like administration. Administration in the form of creams, lotions, tablets, capsules, pellets, dispersible powders, granules, suppositories, syrups, elixirs, lozenges, injectable solutions, sterile aqueous or non-aqueous solutions, suspensions or emulsions, patches, and the like, is also contemplated. The active ingredients may be compounded with non-toxic, pharmaceutically acceptable carriers including, glucose, lactose, gum acacia, gelatin, mannitol, starch paste, magnesium trisilicate, talc, corn starch, keratin, colloidal silica, potato starch, urea, dextrans, and the like.

The preferred route of administration will vary with the clinical indication. Some variation in dosage will necessarily occur depending upon the condition of the patient being treated, and the physician will, in any event, determine the appropriate dose for the individual patient. The effective amount of compound per unit dose depends, among other things, on the body weight, physiology, and chosen inoculation regimen. A unit dose of compound refers to the weight of compound employed per administration event without the weight of carrier (when carrier is used).

Targeted-delivery systems, such as polymer matrices, liposomes, and microspheres can increase the effective concentration of a therapeutic agent at the site where the therapeutic agent is needed and decrease undesired effects of the therapeutic agent. With more efficient delivery of a therapeutic agent, systemic concentrations of the agent are reduced because lesser amounts of the therapeutic agent can be administered while accruing the same or better therapeutic results. Methodologies applicable to increased delivery efficiency of therapeutic agents typically focus on attaching a targeting moiety to the therapeutic agent or to a carrier which is subsequently loaded with a therapeutic agent.

Various drug delivery systems have been designed by using carriers such as proteins, peptides, polysaccharides, synthetic polymers, colloidal particles (i.e., liposomes, vesicles or micelles), microemulsions, microspheres and nanoparticles. These carriers, which contain entrapped pharmaceutically useful agents, are intended to achieve controlled cell-specific or tissue-specific drug release.

The compounds described-herein can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The compounds described herein, when in liposome form can contain, in addition to the compounds described herein, stabilizers, preservatives, excipients, and the like. The preferred lipids are the phospholipids and the phosphatidyl cholines (lecithins), both natural and synthetic. Methods to form liposomes are known in the art. (See, e.g., Prescott, Ed., Methods in Cell Biology, Volume XIV, Academic Press, New York, N.Y., (1976), p 33 et seq.) Several delivery approaches can be used to deliver therapeutic agents to the brain by circumventing the blood-brain barrier. Such approaches utilize intrathecal injections, surgical implants (Ommaya, Cancer Drug Delivery, 1: 169-178 (1984) and U.S. Pat. No. 5,222,982), interstitial infusion (Bobo et al., Proc. Natl. Acad. Sci. USA, 91: 2076-2080 (1994)), and the like. These strategies deliver an agent to the CNS by direct administration into the cerebrospinal fluid (CSF) or into the brain parenchyma (ECF).

Drug delivery to the central nervous system through the cerebrospinal fluid is achieved, for example, by means of a subdurally implantable device named after its inventor the “Ommaya reservoir.” The drug is injected into the device and subsequently released into the cerebrospinal fluid surrounding the brain. It can be directed toward specific areas of exposed brain tissue which then adsorb the drug. This adsorption is limited since the drug does not travel freely. A modified device, whereby the reservoir is implanted in the abdominal cavity and the injected drug is transported by cerebrospinal fluid (taken from and returned to the spine) to the ventricular space of the brain, is used for agent administration. Through omega-3 derivatization, site-specific biomolecular complexes can overcome the limited adsorption and movement of therapeutic agents through brain tissue.

Another strategy to improve agent delivery to the CNS is by increasing the agent absorption (adsorption and transport) through the blood-brain barrier and the uptake of therapeutic agent by the cells (Broadwell, Acta Neuropathol., 79: 117-128 (1989); Pardridge et al., J. Pharmacol. Experim. Therapeutics, 255: 893-899 (1990); Banks et al., Progress in Brain Research, 91:139-148 (1992); Pardridge, Fuel Homeostasis and the Nervous System, ed.: Vranic et al., Plenum Press, New York, 43-53 (1991)). The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier, or by its covalent coupling to a peptide vector capable of transporting the agent through the blood-brain barrier. Peptide transport vectors are also known as blood-brain barrier permeabilizer compounds (U.S. Pat. No. 5,268,164). Site specific macromolecules with lipophilic characteristics useful for delivery to the brain are described in U.S. Pat. No. 6,005,004.

Other examples (U.S. Pat. No. 4,701,521, and U.S. Pat. No. 4,847,240) describe a method of covalently bonding an agent to a cationic macromolecular carrier which enters into the cells at relatively higher rates. These patents teach enhancement in cellular uptake of bio-molecules into the cells when covalently bonded to cationic resins.

U.S. Pat. No. 4,046,722 discloses anti-cancer drugs covalently bonded to cationic polymers for the purpose of directing them to cells bearing specific antigens. The polymeric carriers have molecular weights of about 5,000 to 500,000. Such polymeric carriers can be employed to deliver compounds described herein in a targeted manner.

Further work involving covalent bonding of an agent to a cationic polymer through an acid-sensitive intermediate (also known as a spacer) molecule, is described in U.S. Pat. No. 4,631,190 and U.S. Pat. No. 5,144,011. Various spacer molecules, such as cis-aconitic acid, are covalently linked to the agent and to the polymeric carrier. They control the release of the agent from the macromolecular carrier when subjected to a mild increase in acidity, such as probably occurs within a lysosome of the cell. The drug can be selectively hydrolyzed from the molecular conjugate and released in the cell in its unmodified and active form. Molecular conjugates are transported to lysosomes, where they are metabolized under the action of lysosomal enzymes at a substantially more acidic pH than other compartments or fluids within a cell or body. The pH of a lysosome is shown to be about 4.8, while during the initial stage of the conjugate digestion, the pH is possibly as low as 3.8.

As employed herein, the phrase “therapeutically effective amount,” when used in reference to invention methods employing polycyclic compounds, refers to a dose of compound sufficient to provide circulating concentrations high enough to impart a beneficial effect on the recipient thereof. The specific therapeutically effective dose level for any particular patient will depend upon a variety of factors including the disorder being treated, the severity of the disorder, the activity of the specific compound used, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically fall in the range of about 0.001 up to 100 mg/kg/day; with levels in the range of about 0.05 up to 10 mg/kg/day being preferred.

In accordance with still another embodiment of the invention, there are provided methods for preventing disease conditions in a subject at risk thereof, said methods comprising administering to said subject a therapeutically effective amount of at least one of the compounds described herein.

As used herein, the phrase “preventing disease conditions” refers to averting a disease, disorder or condition from occurring in a subject who may be at risk for the disease, but has not yet been diagnosed as having the disease. Those of skill in the art will understand that a variety of methods may be used to determine a subject at risk for a disease, and that whether a subject is at risk for a disease will depend on a variety of factors known to those of skill in the art, including genetic make-up of the subject, age, body weight, sex, diet, general health, occupation, exposure to environmental conditions, marital status, and the like, of the subject.

As used herein, “incubating” refers to conditions which allow contact between the test compound and the cell of interest. The cell may be any cell of interest including neuronal cells, glial cells, cardiac cells, bronchial cells, uterine cells, testicular cells, liver cells, renal cells, intestinal cells, cells from the thymus and spleen, placental cells, endothelial cells, endocrine cells including thyroid, parathyroid, pituitary, and the like, smooth muscle cells, skeletal muscle cells, and the like.

Candidate compounds can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

A variety of other agents may be included in the screening assay. These include agents like salts, natural proteins, e.g., albumin, detergents, etc. that are used to facilitate optimal binding and/or reduce nonspecific or background interactions. Reagents that improve the efficiency of the assay, such as protease inhibitors, nuclease inhibitors, antimicrobial agents, and the like may be used. The mixture of components can be added in any order that provides for the requisite binding. Incubations are performed at any suitable temperature, typically between 4 and 40° C. Incubation periods are selected for optimum activity, but may also be optimized to facilitate rapid high-throughput screening.

Typically between 0.1 and 10 h will be sufficient.

In accordance with still another embodiment of the present invention, there are provided methods of modulating the aggregation of amyloid proteins, particularly amylin. Modulation of amyloid protein aggregation can prevent or delay the onset of a disease associated with amyloid deposition. In a method of modulating aggregation of amyloid proteins, amyloid proteins are contacted with compounds described herein such that the aggregation of amyloid proteins is altered. As used herein, the term “modulating” refers to both inhibition of amyloid aggregation and promotion of amyloid aggregation. Aggregation of amyloid proteins is inhibited by one or more compounds described herein when there is a decrease in the amount and/or rate of amyloid aggregation in the presence of one or more compounds described herein as compared to the amount and/or rate of amyloid aggregation in the absence of the same one or more compounds. Inhibition of aggregation includes both complete and partial inhibition of amyloid proteins. Inhibition of aggregation can be quantitated as the fold increase in the lag time for aggregation or as the decrease in the overall plateau level of aggregation (i.e., total amount of aggregation), using an aggregation assay known to those of skill in the art. Alternatively, aggregation of amyloid proteins is promoted by one or more compounds described herein when there is an increase in the amount and/or rate of amyloid aggregation in the presence of one more or more compounds described herein compared to the amount and/or rate of amyloid aggregation in the absence of one or more of the same compounds.

As used herein therefore the term “polycyclic compounds” refers to any polycyclic compounds having such effects or an effect. Examples of (but not solely confined to) suitable polycyclic compounds include fused tricyclic compounds, fused four ring compounds, fused five ring structures or other fused polycyclics, and combinations of fused ring and biphenyl structures in planar or non-planar orientations. Various compounds are described below.

The term “polyacene” as used herein refers to a molecular structure generally comprising two or more fused aromatic rings. Polyacenes having three, four, or five fused aromatic rings are preferred. Ring atoms of polyacenes are generally carbon-based, but may also include one or more nitrogens, oxygens, and/or sulfurs. Core structures of polyacenes are substantially flattened, a characteristic that allows extensive overlap of π-electrons between core atoms. Polyacenes of the present invention may be optionally substituted, for example with substituents that enhance aqueous solubility, that enhance π-stacking effects, or that enhance the efficacy of the drug or otherwise improve the efficacy of treatment.

The term(s) “three-, four-, and five-membered ring polyacene(s)” as used herein refers to a molecular structure comprising three, four, and five fused aromatic rings. Ring atoms of three-, four-, and five-membered ring polyacenes are generally carbon, but may also include one or more nitrogens, oxygens, and/or sulfurs. Examples of three-membered ring polyacenes include but are not limited to anthracene, phenalene, phenanthrene, quinacrine, neutral red, chlorpromazine, acridine, acridine orange, methylene blue, phenanthroline, phenazine, and phenothiazine. Examples of four-membered ring polyacenes include pyrene, chrysene, benz[a]anthracene, benz[m]anthracene, and tetracene. A representative examples of a five-membered-ring polyacene is benz[c]anthracene.

Core structures of polyacenes are preferably substantially flattened, a characteristic which allows extensive overlap of π-electrons between core atoms. Polyacenes of the present invention may be optionally substituted, for example with substituents that enhance aqueous solubility, that enhance r-stacking effects, or that enhance the efficacy of the drug or otherwise improve the efficacy of treatment.

Numerous substituted three-, four-, and five-membered ring polyacenes are commercially available. Others may be prepared using methods known to the skilled artisan. Particularly useful reactions for introducing substituents onto three-, four-, and five-membered ring polyacenes are classes of reactions known as electrophilic aromatic substitution. Examples of electrophilic aromatic substitution include: Friedel-Crafts alkylation (useful for attaching alkyl groups to one or more sites on a three-, four-, and/or five-membered ring polyacene; Friedel-Crafts acylation (useful for covalently attaching carboxylate groups to one or more sites on a three-, four-, and/or five-membered ring polyacene; nitrosation (useful for introducing a nitroso moiety (—NO) to one or more sites on a three-, four-, and/or five-membered ring polyacene; sulfonation (useful for introducing the sulfate moiety (—SO₃⁻) to one or more sites on a three-, four-, and/or five-membered ring polyacene; nitration, useful for inducing the nitrate (—NO₂) moiety which can also be reduced halogenation, useful for introducing F, Cl, Br, and I; diazo coupling, useful for coupling aromatic rings linked by a diazo (—N═N—) moiety as found for example in Congo red, chrysamine G, and amaranth.

The skilled artisan will recognise that several moieties introduced to three-, four-, and/or five-membered ring polyacenes via methods of electrophilic aromatic substitution may be reduced. For example, nitro and nitroso groups may be reduced to amino groups, sulfonate groups may be reduced to thiols. Furthermore the skilled artisan will recognise that several such moieties introduced into three-, four-, and/or five-membered ring polyacenes are themselves reactive and useful functional groups. For example, amino and carboxylate moieties useful precursors for amide linkages; thiol moieties are exceptionally useful linker moieties.

The skilled artisan will also recognise partially reduced polyacenes within other embodiments of the present invention, for example, the tetracene framework within the molecules tetracycline and doxycycline.

### Three-Membered Ring Polyacenes

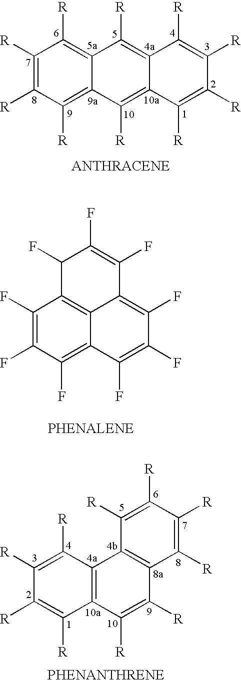

R (which in any compound can be the same or different, and can include H) represents moieties introduced using methods known to the skilled artisan, for example methods known as electrophilic aromatic substitution. The structures shown above also represent the case where one, some or all R of a compound is hydrogen. Fused tricyclic compounds also include derivatives with substitutions at any of the core atoms numbered as shown above. Also included are modifications of the double bond structure within the core tricyclic ring structure and subsequent core atom or side chain modifications, including those at atoms: 4a, 4b, 5a, 8a, 9a and 10a as illustrated for anthracene and phenanthrene. Representative examples are quinacrine, neutral red, chlorpromazine, acridine, acridine orange, methylene blue, and phenodiazine (as below). While polyacene molecules are generally substantially planar, the skilled artisan recognizes that partial reduction of polyacene introduces non planarity. Thus fused ring compounds of the present invention may be planar or non-planar and may have any combination of saturated or unsaturated ring structures. Structures may also possess any of the phenyl rings placed in any alternative orientation to that shown.

### Substituted And Non-Substituted Three-Membered Ring Polyacenes

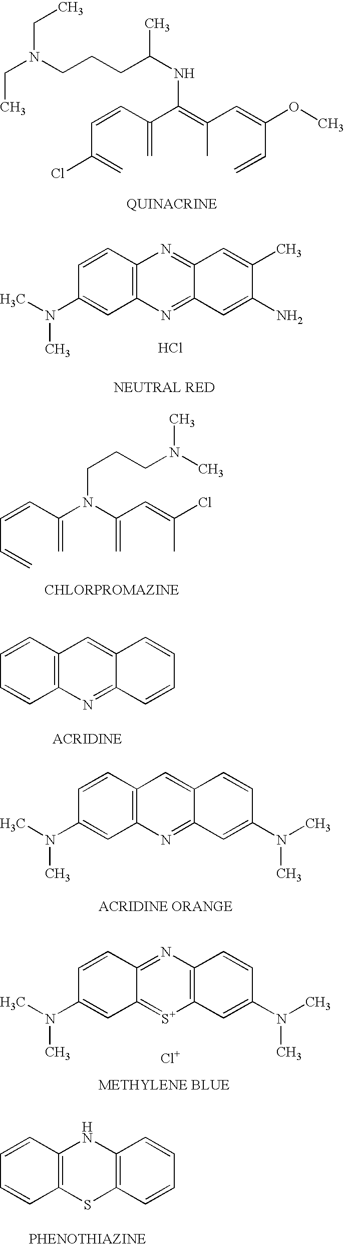

### Four- and Five-Membered Ring Polyacenes

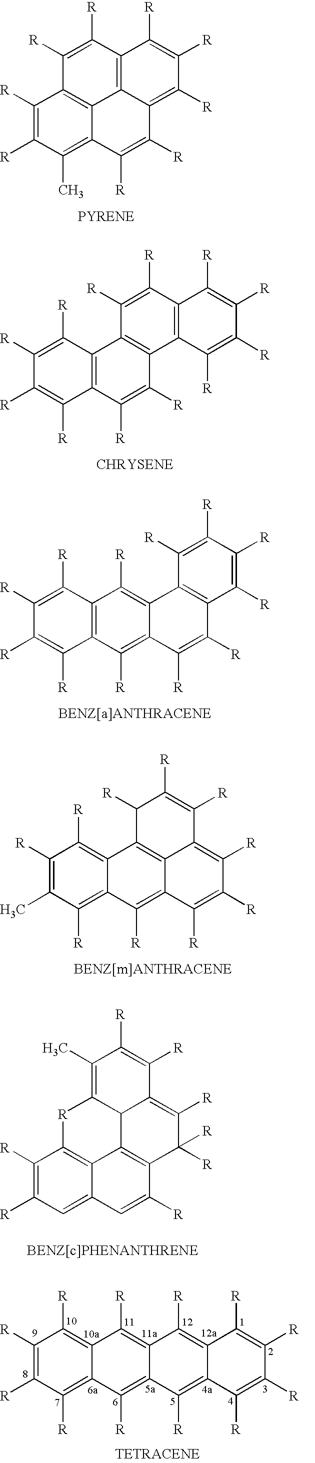

R (which in any compound can be the same or different, and can include H) represents moieties introduced using methods known to the skilled artisan, for example methods known as electrophilic aromatic substitution. The structures shown above also represent the case where one, some or all R of a compound is hydrogen. Fused anthracyclic structures also include derivatives with substitutions at any of the core atoms numbered as shown. Also included are modifications of the double bond structure within the core anthracyclic ring structure and subsequent core atom or side chain modifications, including those at atoms: 4a, 5a, 6a, 10a, 11a and 12a, as illustrated with tetracene above. While polyacene molecules are substantially planar, the skilled artisan recognizes that partial reduction of polyacene introduces non planarity. Thus fused ring compounds of the present invention may be planar or non-planar and may have any combination of saturated or unsaturated ring structures. Structures may also possess any of the phenyl rings placed in any alternative orientation to that shown. Representative examples are tetracycline and doxycycline.

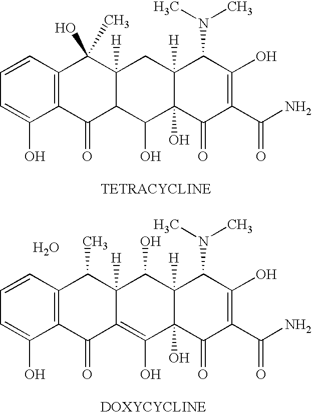

### Representative Examples of Fused Ring and Biphenyl Structure Combinations

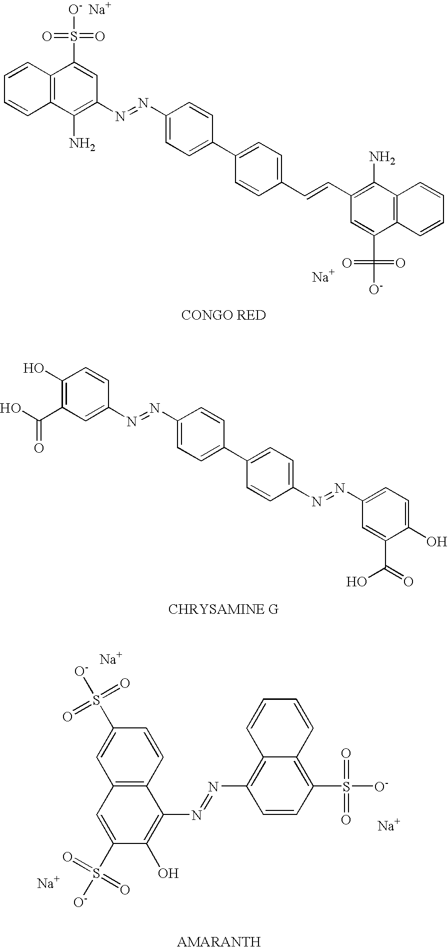

Structures may also possess any combination of fused and biphenyl rings and any of the phenyl rings placed in any alternative orientation to that shown. These structures may be either planar or non-planar, symmetrical or non-symmetrical, and may have any combination of saturated or unsaturated ring structures. Structures may also possess any of the phenyl rings placed in any alternative orientation to that shown. These structures also include derivatives with substitutions at any of the aromatic ring atoms. Also included are modifications of the double bond structure within the ring structures and subsequent atom or side chain modifications.

The following examples are intended to illustrate but not to limit the invention in any manner, shape, or form, either explicitly or implicitly. While they are typical of those that might be used, other procedures, methodologies, or techniques known to those skilled in the art may alternatively be used.

### EXAMPLE 1

### Materials And Methods

Materials. Synthetic human (Lot 5429559 and Lot 0551805) and rat amylin (Lot 0542554 and Lot 0542554) were HPLC-purified products from Bachem Calif. (Torrance, Calif.). Peptides were freshly dissolved in sterile milliQ water then diluted to their final concentration in the appropriate buffer. Tritated human amylin (145.3 MBq/mmol) and rat amylin (22.6 GBq/mmol) were synthesised according to protocols as described previously (25).All incubations containing amylin peptides were carried out at 22° C. All polycyclic compounds and thioflavin-T were purchased from Sigma (St Louis, Mo.). Stock solutions were made fresh in sterile milliQ water for each experiment. Calcein-AM and ethidium homodimer-1 (EthD-1) were obtained from Molecular Probes (Eugene, Oreg., USA). The rat insulinoma cell line RINm5F was obtained from the National Institutes of Health, Bethesda, Md. and cultured at 37° C. in a humidified atmosphere containing 5% CO₂. Cell culture medium and its supplements were purchased from GibcoBRL-Life Technologies (Auckland, New Zealand).

Thioflavin-T binding fluorescent assays. The effects of various polycyclic compounds on islet amyloid formation were measured by fluorescence spectroscopy, using a SpectraMAX Gemini XS Fluorescence Spectrophotometer (Molecular Devices Corporation, Sunnyvale, Calif.). Excitation and emission maxima were set to 450 nm and 510 nm respectively using a cutoff filter at 495 nm. Thioflavin-T binds to amylin fibrils, but not to monomeric amylin (Goldsbury et al., (1999), J. Mol. Biol., 285:33-39). When bound to amylin fibrils, thioflavin-T shows a marked increase in fluorescence that can be quantitated using a fluorescence spectrophotometer (Goldsbury et al., (1999), J. Mol. Biol., 285:33-39). The rate of amylin fibril formation was determined by following thioflavin-T fluorescence in the presence or absence of the potential inhibitory drug. The polycyclic compounds tetracycline, congo red, neutral red, methylene blue and chlorpromazine have no intrinsic fluorescence under these conditions. Background fluorescence by acridine and acridine orange in the absence of amylin was subtracted from the experimental results. The control preparation contained human amylin and thioflavin-T in the absence of drug. The rate of fluorescence enhancement under these conditions was used to compare amylin fibril formation in the absence and presence of the drug. All other experimental conditions were identical.

Tri-prolyl amylin and rat amylin with thioflavin-T were also used as additional controls. Tri-prolyl amylin is a modified form of amylin, which no longer contains the amyloidogenic region and thus is unable to form fibrils (Evans & Krentz, (1999), Drugs R D, 2:75-94), while rat amylin does not spontaneously form fibrils (Cooper, (1994), Endocr. Rev., 15:163-201).

Electron Microscopy. Human amylin was incubated in 10 mM tris pH 7.4 solution in the presence or absence of either quinacrine or tetracycline. Samples were removed at various time points and prepared for electron microscopy. Aliquots (8 μl) of the amylin fibril preparations were absorbed to glow-discharged carbon-coated collodion film on 200-mesh copper grids for 1 min. Grids were blotted, washed twice in droplets of deionised water and stained with 2% (w/v) uranyl acetate. Grids were examined in a Phillips Technai transmission electron microscope operated at 120 kV.

Radiolabelled Precipitation Assays. The precipitation of radiolabelled human and rat amylin fibrils was used as an independent method to monitor assembly of amylin fibrils in the presence or absence of potential inhibitors of amylin fibril formation. Trace amounts of [³H]-human amylin were added to a 10 μM amylin solution in the absence or presence of 100 μM tetracycline (Example 5 herein) or 200 μM potential inhibitory drug (Example 5 herein) for various times. Incubation mixtures were then centrifuged at either 16,000×g or 100,000×g, for 20 min, and the amount of [³H]-human amylin remaining in the supernatant after centrifugation was determined (Beckman LSW 3801 β-counter, USA). Results were expressed as percentages of precipitable counts per minute, relative to total radioactivity in the supernatant.

Preparation of monomeric human amylin. Human amylin (batch 0551805) was obtained from Bachem (Torrence, Calif.). Stock solutions of amylin were prepared as described by Padrick and Miranker, 2002. Human amylin was solubilised in 6M guanidine HCL/50 mM potassium phosphate, pH 6.0, and loaded onto a C18 reversed-phase spin column (Harvard Biosciences). The column was then washed sequentially with 10% Acetonitrile, 0.2% trifluoroacetic acid and water. Monomeric amylin was eluted in 100% HFIP (hexafluoroisopropanol). This stock solution of amylin was then used for all Circular Dichroism experiments.

Circular Dichroism Assay. Circular dichroism spectra were measured on a Pi-Star 180 spectrometer (Applied Photophysics, Leatherhead, UK). Measurements were carried out in either 100% HFIP or 100 mM potassium chloride/50 mM potassium phosphate buffer (pH 7.4) and 2.5% HFIP at 25° C. A stock solution of monomeric human amylin was diluted into buffer to a final concentration of 2.5% HFIP and approximately 5 μM amylin to initiate amylin fibril formation. Spectra were collected at 1 nm intervals with a sample period of 25 μs and adaptive sampling of ±0.01 mdeg. Measurements were recorded immediately following addition of the human amylin stock solution to phosphate buffer in the presence or absence of Congo red or amaranth.

Cell Culture and Cytotoxicity Assays. RINm5F cells were cultured in RPMI 1640 medium containing 10% fetal bovine serum, 290 μg/mL L-glutamine, 100 IU/mL penicillin and 100 μg/mL streptomycin. Cells were plated at a density of 15×1 cells per well, incubated for 48 h, rinsed with PBS and placed in fresh medium containing Congo red. Freshly prepared human amylin in Milli-Q water, was preincubated with Congo red for 30 min, then added to the cell culture medium to give a final concentration of 30 μM. Cells were treated for 22 h with human amylin in the presence or absence of 100 μM Congo red. Cell viability was determined by double-staining with calcein-AM and ethidium homodimer-1. Green fluorescence of live cells and red fluorescence marking nuclei of dead cells were simultaneously visualized using a Zeiss Axiovert S100 microscope equipped with a Zeiss filter set # 09. Photographs were taken at 400× magnification using a Zeiss AxioCam digital camera.

### EXAMPLE 2

### Effects of Polycyclics on Enhancement of Thioflavin-T Fluorescence by Human Amylin

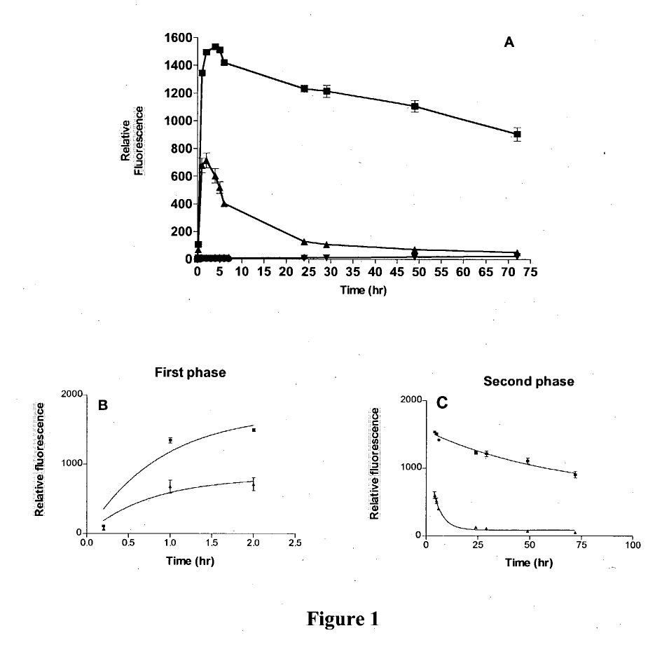

Effect of tetracycline and congo red on enhancement of thioflavin-T fluorescence by human amylin (FIG. 1). Tetracycline (700 μM) (π) or congo red (700 μM) (●)were incubated in 10 mM tris pH 7.4, with monomeric human amylin (70 μM) in the presence of thioflavin-T (10 μM).The fluorescence of the reaction was monitored over 72 hours as described in the methods. A control preparation, containing human amylin and thioflavin-T only (▪), was used to compare amylin fibril formation, in the absence of the drugs, with fibril formation when either tetracycline or congo red was present. Tri-prolyl amylin (θ) and rat amylin (♦) with thioflavin-T served as additional controls, since neither tri-prolyl or rat a mylin spontaneously form fibrils under these assay conditions. Each data point represents the mean±s.e.m of three separate reactions.

In the presence of tetracycline, there is an initial but significantly decreased formation of a mylin fibril-associated fluorescence up to 2 h ours, followed by a gradual decrease in fluorescence over the next 70 hours (FIG. 1A). This decrease in fluorescence correlates with a breakdown of amylin fibrils as seen by electron microscopy. The initial phase of association of thioflavin-T with human amylin in the presence of tetracycline (FIG. 1B), is similar but lower than that of thioflavin-T alone with human amylin. However, there is a marked second dissociation phase of thioflavin-T from amylin fibrils in the presence of tetracycline, that is not seen in the control reaction (FIG. 1C). This correlates with the disruption of amylin fibrils at 24 hours and beyond as seen under the electron microscope. Human amylin in the presence of congo red and thioflavin-T, shows no increase in fluorescence beyond that of the rat control preparation, suggesting that congo red may inhibit amylin-fibril formation or binding of thioflavin-T to amylin fibrils.

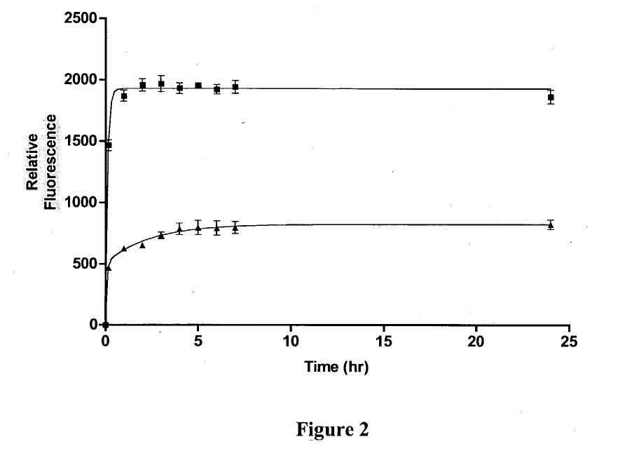

Effect of chlorpromazine on enhancement of thioflavin-Tfluorescence by human amylin (FIG. 2). Chlorpromazine (700 μM) (π) was incubated in 10 mM tris p H 7.4, with monomeric human amylin (70 μM) in the presence of thioflavin-T (10 μM). The fluorescence of the reaction was monitored over 24 hours as described in the methods. The curve showing the control containing only human amylin and thioflavin-T (▪), was fitted according to a single phase association, while the curve of human amylin and thioflavin-T in the presence of chlorpromazine was fitted with a two phase exponential association. Each data point represents the mean±s.e.m of three separate reactions.

In the presence of chlorpromazine, there is approximately 60% inhibition of amylin fibril formation. The best-fit curve for the results of the experiment containing human amylin, thioflavin-T and chlorpromazine shows an initial exponential association phase of thioflavin-T to human amylin, similar to that seen in the control containing only human amylin and thioflavin-T. However, this is followed by a second slower exponential association phase not seen in the control, which shows an inhibitory effect of chlorpromazine on amylin fibril formation.

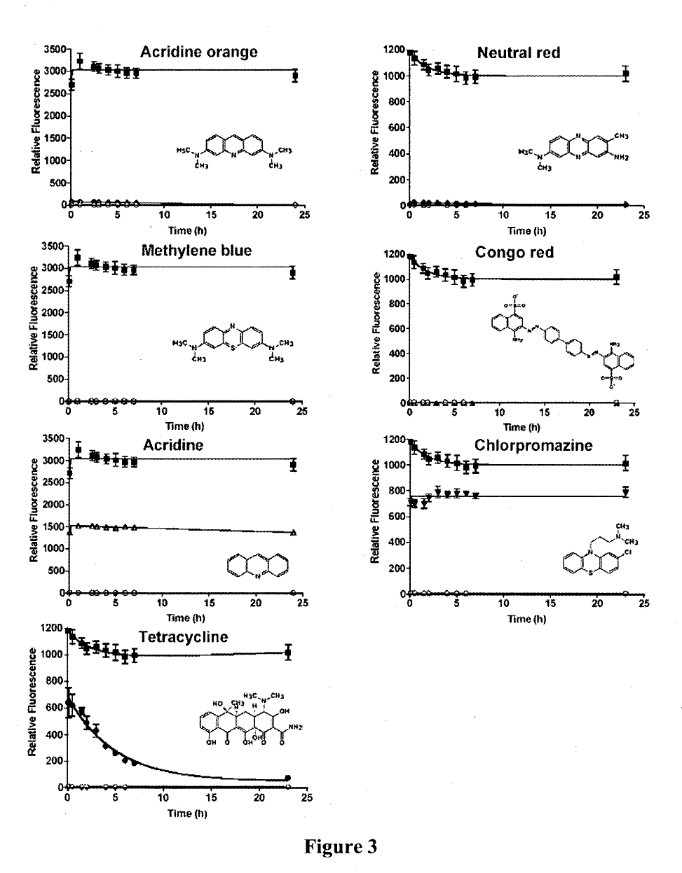

Effect of selected polycylic compounds on enhancement of thioflavin-Tfluorescence by human amylin (FIG. 3). Acridine, acridine orange, neutral red and methylene blue are selected examples of fused tricyclic ring compounds. Tetracycline is a fused four-ring compound whereas Congo red is a combination of two paired fused rings with intervening biphenyl structures. Compound structures are shown adjacent to the appropriate graph. Acridine orange (⋄), neutral red (♦), methylene blue (□), Congo red (π), acridine (ρ), chlorpromazine (θ) and tetracycline (●), at final concentrations of 1200 μM, were incubated in 10 mM tris pH 7.4, with monomeric human amylin (60 μM) in the presence of thioflavin-T (10 μM). Fluorescence was monitored over 24 h as described in the Methods. A preparation containing human amylin and thioflavin-T only (▪), was included as a comparison in the absence of drug. Rat amylin (∘) with thioflavin-T served as a negative control in each experiment, since rat amylin does not spontaneously form fibrils. Each data point represents the mean±s.e.m of three independent experiments.

Incubation of human amylin with thioflavin-T in the presence of a 20-fold molar excess of either acridine orange, neutral red, methylene blue or Congo red showed no increase in thioflavin-T enhanced fluorescence, compared with an immediate and sustained increase in fluorescence when human amylin was incubated with thioflavin-T alone. This relative reduction in fluorescence could be due to displacement of thioflavin-T binding to existing amyloid, or alternatively, to a direct reduction in amyloid content caused by the presence of these polycyclic compounds.

When tetracycline was incubated with human amylin and thioflavin-T, an immediate increase in fluorescence occurred, followed by a gradual decrease in fluorescence over the next 24 h. The initial phase of association of thioflavin-T with human amylin in the presence of tetracycline was similar but significantly lower than that of thioflavin-T and human amylin alone. However when tetracycline was present, there was a marked second dissociation phase with a half-life of 3.4 hours that was not seen in the control reaction.

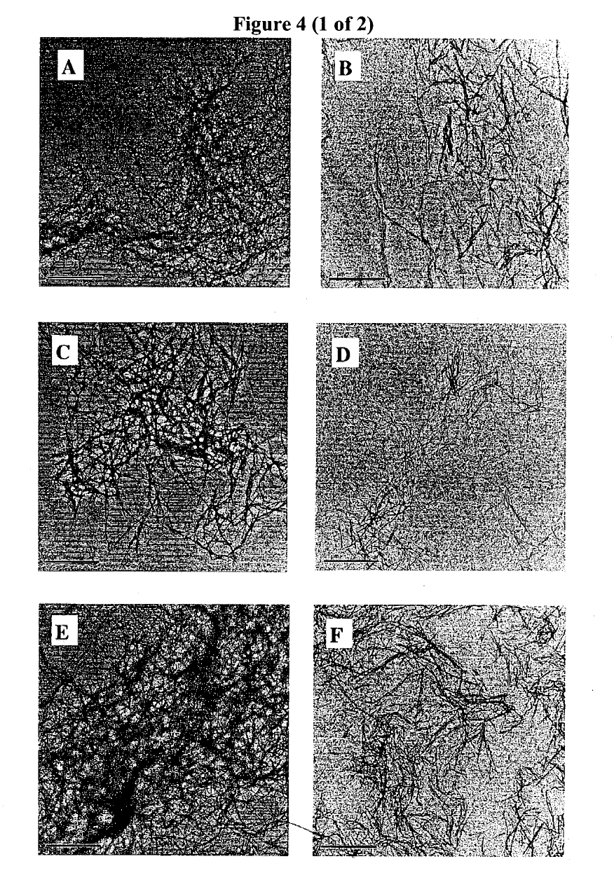

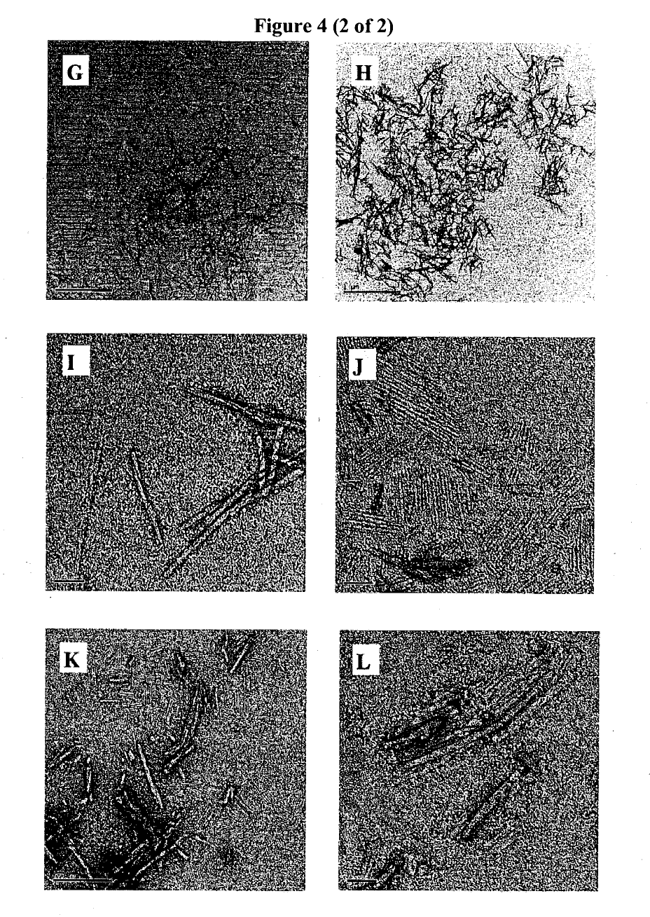

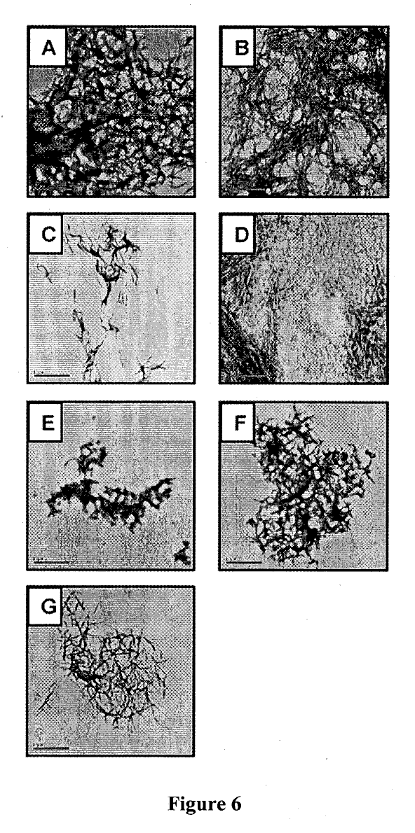

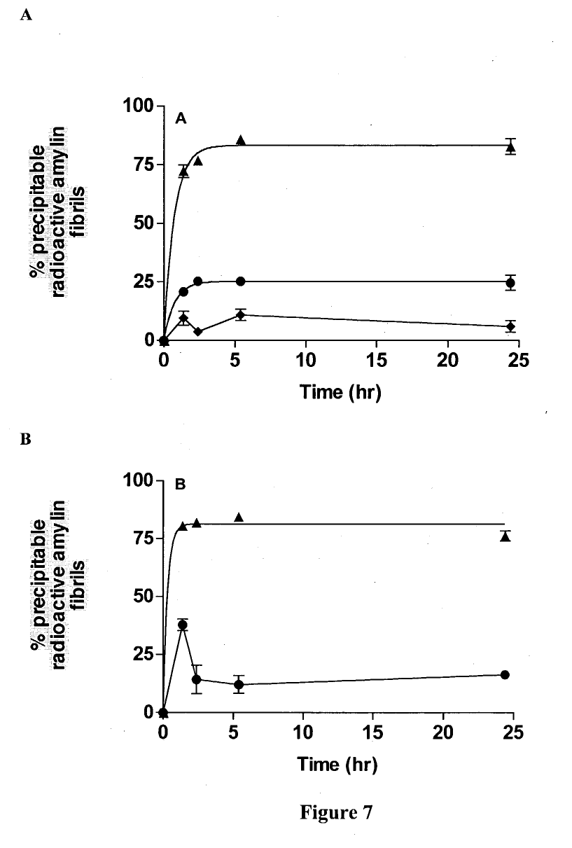

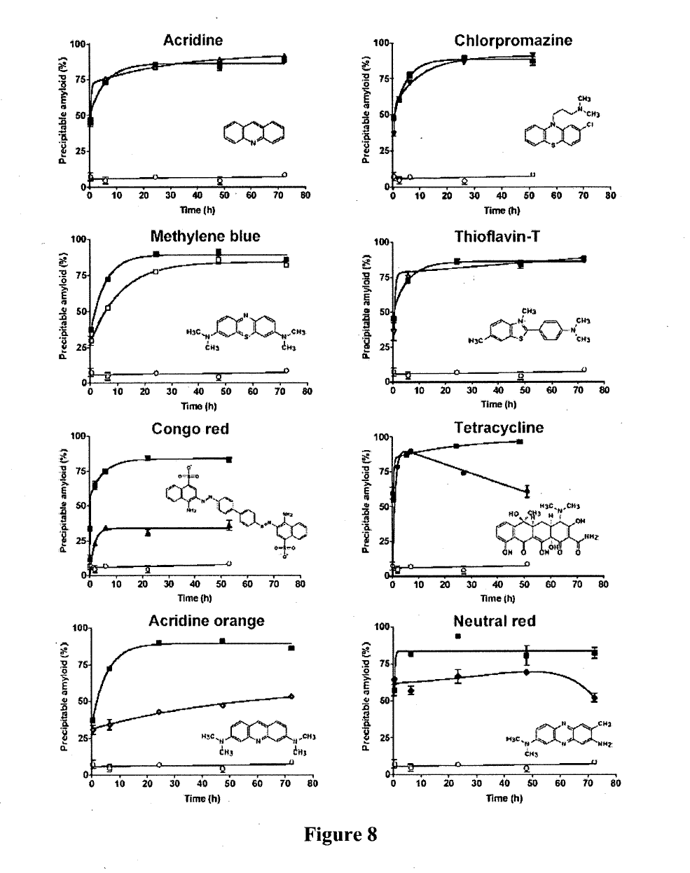

This change correlates with decreases in precipitable amyloid content (FIGS. 7 and 8) and alterations in the morphology of amyloid fibrils at 24 h as observed by electron microscopy (FIGS. 4C, 4D, 6C and 6D). Chlorpromazine, a tricyclic compound which contains an aliphatic sidechain on the central aromatic ring, showed little inhibition of amylin fibril associated thioflavin-T fluorescence enhancement, indicating that this compound, in contrast to acridine orange, neutral red, methylene blue, Congo red and tetracycline, does not effectively displace thioflavin-T or suppress amyloid formation. Acridine, which contains only the parental phenazine structure, by contrast showed a moderate reduction in thioflavin-T enhanced fluorescence.

### EXAMPLE 3

### Effects of Polycyclics on Human Islet Amyloid Formation as Measured by Electron Microscopy

Electron Microscopy of human amylin fibrils in the presence and absence of tetracycline (FIG. 4). A 10 fold molar excess of tetracycline was incubated with human amylin. Samples were removed at various time points and prepared for electron microscopy according to the procedure described in the methods.

1. 70 μM human amylin after 1.5 h incubation. Magnification: ×20,500

2. 70 μM human amylin+700 μM tetracycline after 1.5 h incubation. Magnification: ×20,500

3. 70 μM human amylin after 5 h incubation. Magnification: ×20,500

4. 70 μM human amylin+700 μM tetracycline after 5 h incubation. Magnification: ×20,500

5. 70 μM human amylin after 29 h incubation. Magnification: ×20,500

6. 70 μM human amylin+700 μM tetracycline after 29 h incubation. Magnification: ×20,500

7. 70 μM human amylin after 48 h incubation. Magnification: ×20,500

8. 70 μM human amylin+700 μM tetracycline after 48 h incubation. Magnification: ×20,500

9. 70 μM human amylin after 29 h incubation. Magnification: ×220,000

10. 70 μM human amylin+700 μM tetracycline after 29 h incubation. Magnification: ×220,000

11. 70 μM human amylin+700 μM tetracycline after 48 h incubation. Magnification: ×105,000

12. 70 μM human amylin+700 μM tetracycline after 48 h incubation. Magnification: ×220,000

The presence of tetracycline appears to slow the rate of formation of human amylin fibrils at the early timepoints of 1.5 hours and 5 hours incubation (4B, 4D), compared to the human amylin control incubated without tetracycline (4A, 4C). At the later timepoints of 29 hours and 48 hours, there appears to be a change in the morphology of the human amylin fibrils, as well as the rate at which fibrils are formed in the presence of tetracycline. The morphology is characterised by short fragmented fibrils (4F, 4H), compared to the longer, more dense and characteristic amylin fibril appearance of the respective controls (4E, 4G). At higher magnification, a different type of fibril structure, which we term a protofibril (lighter stained fibrils), is seen along with the short fragments of fibrils (4J-4L). These protofibrils are not observed at high magnification in the amylin control (4I). The presence of these protofibrils suggests disruption of existing insoluble amylin fibrils.

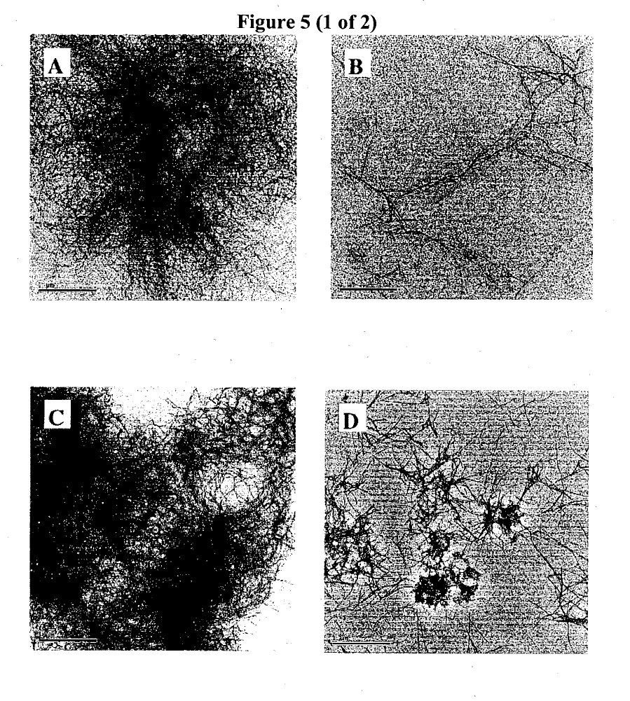

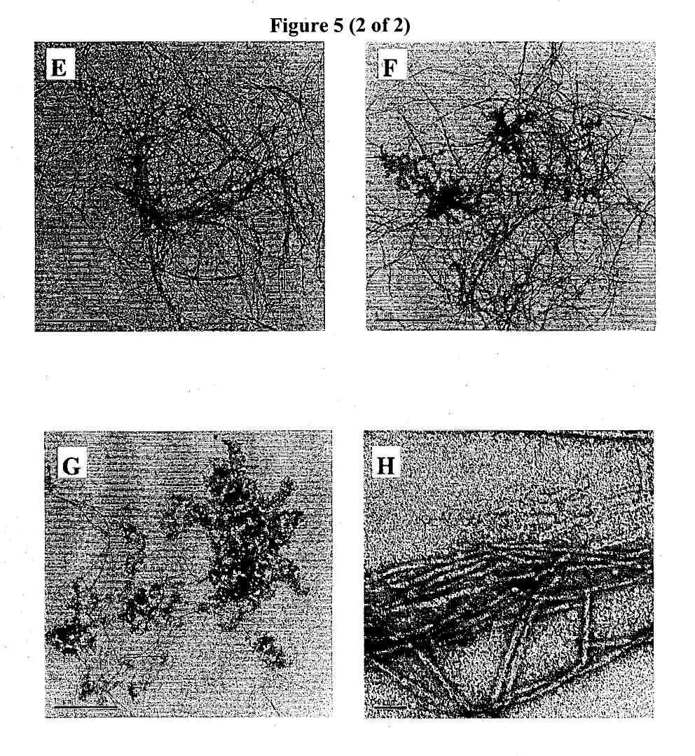

Electron Microscopy of human amylin fibrils in the presence and absence of quinacrine (FIG. 5). A 100 fold molar excess of quinacrine was incubated with human amylin. Samples were removed at various time points and prepared for electron microscopy according to the procedure described in the methods.

A. 70 μM human amylin after 1.5 h incubation. Magnification: ×20,500

B. 70 μM human amylin+7000 μM quinacrine after 1.5 h incubation. Magnification: ×20,500

C. 70 μM human amylin after 5 h incubation. Magnification: ×20,500

D. 70 μM human amylin+7000 μM quinacrine after 5 h incubation. Magnification: ×20,500

E. 70 μM human amylin after 26 h incubation. Magnification: x 20,500

F. 70 μM human amylin+7000 μM quinacrine after 26 h incubation. Magnification: ×20,500

G. 70 μM human amylin+7000 μM quinacrine after 26 h incubation. Magnification: ×20,500

H. 70 μM human amylin+7000 μM quinacrine after 26 h incubation.

I. Magnification: ×220,000

The presence of quinacrine appears to slow the rate of human amylin fibril formation at all three timepoints of 1.5 hours, 5 hours and 26 hours incubation (4B, 4D, 4F), compared to the human a mylin control incubated without quinacrine (4A, 4C, 4E). There also appears to be a difference in the morphology of the fibrils at 5 hours and 26 hours (4D, 4F, 4G), where the amylin fibrils have formed large aggregates with few defined fibrils (4F, 4G) rather than the characteristic longer fibrils seen in the control (4E). The short fragmented fibrils seen with human amylin in the presence of tetracycline, were not observed with human amylin and quinacrine. However, protofibrils may be present (lightly stained fibrils in 4H) in the 26 hour incubation time point. These protofibrils are also seen in the electron micrographs of amylin incubated with tetracycline.

Electron Microscopy of human amylin fibrils in the presence and absence of selected polycyclic compounds (FIG. 6). Human amylin (60 μM) was incubated in 10 mM tris pH 7.4 for 24 h in the presence or absence of either tetracycline, Congo red, neutral red or chlorpromazine (1200 μM). Samples were removed and prepared for electron microscopy as described in the Methods. All experiments were performed in triplicate and the pictures above are representative of at least 6 photographs taken at each magnification. Human amylin (A×20,500, B×105,000); human amylin incubated with tetracycline (C×20,500, D×105,000); human amylin incubated with Congo red (E×20,500); human amylin incubated with neutral red (F×20,500); human amylin incubated with chlorpromazine (G×20,500).

Transmission electron microscopy of human amylin incubated with tetracycline for 24 h, showed a marked change in the morphology of the resulting amyloid fibrils (FIG. 6). Here, fibril morphology was characterized by short fragmented structures (FIG. 6C), compared to the longer, more dense and characteristic amylin fibril appearance of the respective control (FIG. 6A). At higher magnification, small globular lightly stained structures, were seen along with short fragments of fibrils (FIG. 6D). These globular structures were not observed at the higher magnification in the amylin control (FIG. 6B). The presence of these structures suggests disruption of existing amylin fibrils after incubation with tetracycline. Interestingly, the globular structures were not observed when human amylin was incubated with other polycyclic compounds. Human amylin, when incubated with Congo red (FIG. 6E), neutral red (FIG. 6F) or chlorpromazine (FIG. 6G), still formed characteristic amylin fibrils. Additional experiments, where human amylin preparations were pre-incubated for 20 h, followed by incubation with tetracycline for 48 h, also revealed fragments of amylin fibrils interdispersed with smaller fibrils similar to those seen in FIGS. 6C, 6D.

### EXAMPLE 4

### Effects of Polycyclics on Human Islet Amyloid Formation as Measured by Radioactive Precipitation

Effect of tetracycline on amylin fibril formation by radiolabelled precipitation (FIG. 7) 10 μM human amylin in 10 mM tris pH 7.4, was incubated with trace amounts of [³H]-human amylin in the absence (π) or presence (●) of 100 μM tetracycline. Incorporation of [³H]-amylin into amylin fibrils was followed for 24 hours. Human amylin fibrils were precipitated by centrifugation at either 16,000×g (FIG. 7A) or at 100,000×g (FIG. 7B). Rat amylin (10 μM) in the presence of [³H]-rat amylin was used as a non-fibril forming control (♦) (FIG. 7A). The results are shown as the percentage of precipitable radioactive amylin, relative to total radioactivity in the supernatant at each time point. Each data point represents the mean±s.e.m of three separate reactions.

The incorporation of radioactive monomeric amylin into the human amylin fibrils as they form, can be used to measure the rate at which amylin fibrils form in the presence or absence of a potential inhibitory drug. Amylin fibrils can be separated from solution by centrifugation and the amount of radioactive amylin precipitated in the amylin fibril pellet is then used as a measure of the amount of amylin fibrils present at that time.

FIG. 5A shows that if tetracycline is absent, then approximately 75% of the total radioactive human amylin available, is incorporated into precipitable amylin fibrils after 2 hours incubation. However, in the presence of tetracycline, there is approximately a 50% reduction in the incorporation of radioactive human amylin into precipitable amylin fibrils. Moreover, this reduction does not increase over time. The percentage of tetracycline precipitable amylin fibrils, was also unaffected by centrifugation of the reaction mixtures at 100,000×g (FIG. 7B). These observations show that inhibition of islet amyloid formation in the presence of tetracycline is due to a reduction in insoluble amylin aggregates.

Effect of selected polycyclics on amylin fibril formation by radiolabelled precipitation (FIG. 8) 10 μM human amylin with added [³H]-human amylin in 10 mM tris pH 7.4, was incubated in the absence (▪) or presence of 200 μM acridine (ρ), chlorpromazine (θ), methylene blue (□), thioflavin-T (σ), congo red (π), tetracycline (●), acridine orange (⋄), or neutral red (♦).pound structures are shown adjacent to the appropriate graph. Thioflavin-T, although not polycyclic, is widely used in fluorescent assays to measure amylin fibril formation. Incorporation of radioactivity into the pellet of an aliquot of the reaction mixture centrifuged at 16,000×g, for 20 minutes was monitored over the time periods indicated. Rat amylin (10 μM) with [³H]-rat amylin added was used as a non fibril forming control (∘) in each experiment. The results are shown as the percentage of precipitable radioactive amyloid, relative to total radioactivity in the supernatant at each time point. Each data point represents the mean±s.e.m of three separate experiments.

These precipitation experiments showed clearly that the tricyclic compounds acridine, chlorpromazine and methylene blue, had no effects on amyloid formation, as did thioflavin-T. Conversely, Congo red showed significant and rapid inhibition with an approximately 3-fold reduction in amyloid content after 5 h that was sustained over the incubation period. In the absence of Congo red, human amylin showed greater than 75% incorporation of radiolabelled amylin into precipitable amyloid after 5 h incubation.

Acridine orange also showed a significant reduction in formation of precipitable amyloid, to a level of 50% after 72 h, giving an overall 30% decrease. Neutral red, another tricyclic molecule similar in structure to acridine orange, showed a small but significant reduction. Tetracycline also exerted significant inhibition but only after a 50 h incubation period, during which the percentage decreased from approximately 85% to 60%. The mode of inhibition in this case reflected that seen in the thioflavin-T fluorescent experiments, where initial formation of amyloid was followed over time by a tetracycline-dependent dissociation phase.

Methylene blue is an example of a compound which shows complete inhibition of fibril associated thioflavin-T fluorescence, but has no effect on precipitable amyloid content indicating that binding to existing amyloid and/or displacement of thioflavin-T, does not necessarily correlate with inhibition of amyloid formation.

### EXAMPLE 5

### Inhibition of Human Amylin Fibril Formation by Polycyclics as Measured by Circular Dichroism

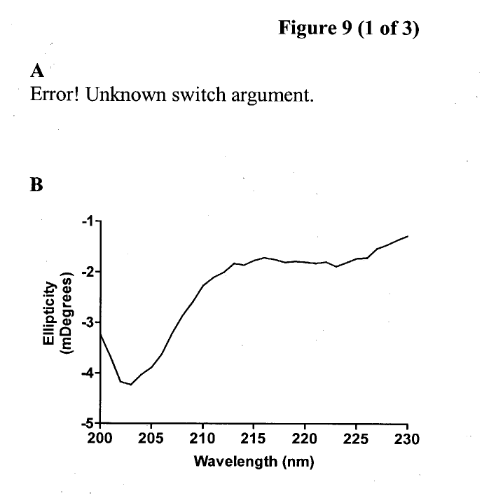

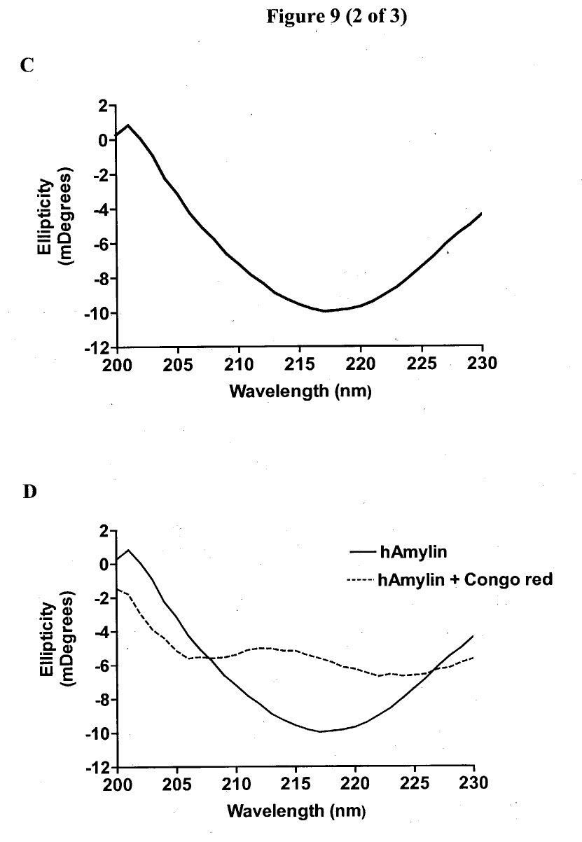

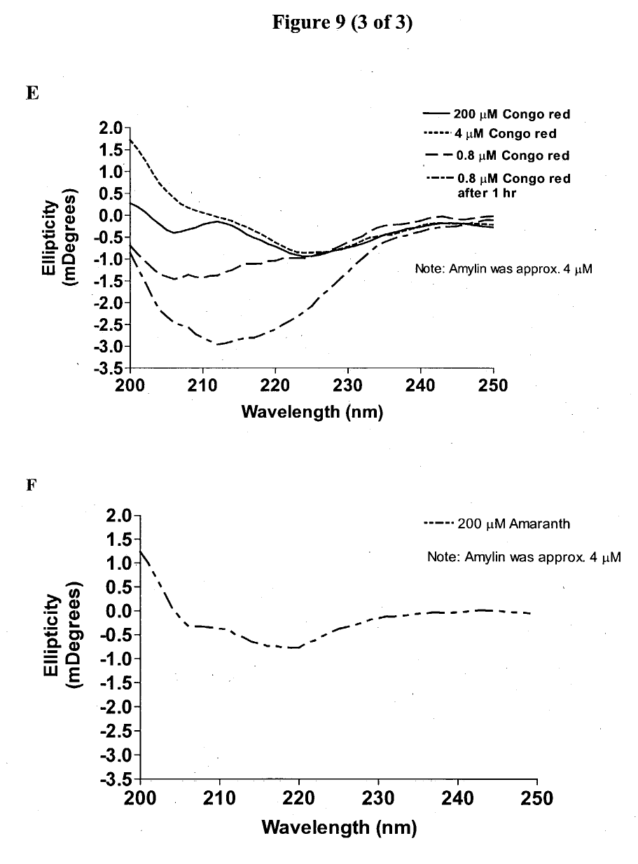

Effect of Congo red on a mylin fibril formation by circular dichroism (FIG. 9) The data outlined below shows the effect of the polycyclic compound Congo red, on the inhibition of a mylin fibril formation using the spectrophotometric technique of Circular Dichroism, using for comparison the Circular Dichroism spectra of pure secondary structures (FIG. 9A). Human amylin (100 μg) was purified on a C18 spin column to give a stock solution in 100% HFIP as described in the methods. A spectrum of this stock solution was collected at 1 nm intervals in a 0.1 cm path length quartz cell (FIG. 9B). The stock solution of monomeric amylin in 100% HFIP was diluted to 2.5% HFIP in 100 mM potassium chloride/50 mM potassium phosphate buffer (pH 7.4). Human amylin concentration was approximately 5 μM. A spectrum was collected at 1 nm intervals in a 0.5 cm path length quartz cell in the absence (FIG. 9C) or presence of 20-fold molar excess of Congo red over human amylin (FIG. 9D). The stock solution of monomeric amylin in 100% HFIP was diluted to 4% HFIP in 100 mM potassium chloride/50 mM potassium phosphate buffer (pH 7.4) containing decreasing concentrations of Congo red as indicated in the Figure. Human amylin concentration was approximately 4 μM. Spectra were collected at 1 nm intervals in a 0.1 cm path length quartz cell (FIG. 9E). The stock solution of monomeric amylin in 100% HFIP was diluted to 4% HFIP in 100 mM potassium chloride/50 mM potassium phosphate buffer (pH 7.4) containing 200 μM Amaranth. Human amylin concentration was approximately 4 μM. Spectra were collected at 1 nm intervals in a 0.1 cm path length quartz cell (FIG. 9F).

Purified amylin is stabilised in a random coil conformation, when maintained in a solution of 100% HFIP (FIG. 9B). The solution is stable in the conformation over a period of days at room temperature. Rat amylin which does not form fibrils shows a similar spectrum in aqueous solution. However, upon dilution of human amylin from the stock solution into a phosphate-salt buffer, β-sheet formation occurs immediately as seen by the characteristic minimum at 217 nm (FIG. 9C). The formation of β-sheet conformers of human amylin, is the first step in insoluble amylin fibril formation.

In contrast, dilution of amylin into buffer containing an excess of Congo red prevents formation of β-sheet conformers (FIG. 9D) and instead arrests the amylin peptide in an α-helical conformation (minima at 205 and 223 nm). Amylin remains stable in this α-helical structure for at least 24 hr and does not proceed to insoluble fibril formation. Titration of decreasing concentrations of Congo red against human amylin (FIG. 9E) shows a change from α-helix (200 μM and 4 μM Congo red) through to random coil (0.8 μM Congo red) at substoichiometric concentrations of Congo red, which then forms β-sheet (0.8 μM Congo red after 1 hr). It appears that Congo red binds to amylin in stoichiometric ratios to prevent formation of 1-sheet.

Congo red is an example of a polycyclic compound which, under the conditions described, appears to arrest amylin in an α-helical conformational state and prevent the progression to β-conformer formation and thence to insoluble fibril formation.

Amaranth is a food dye related in structure to Congo red and has a similar effect to Congo red on amylin in that an excess of Amaranth prevents formation of β-conformers and instead arrests the amylin peptide in an α-helical conformation.

### EXAMPLE 6

### Protective Effects of Polycyclics Against Amylin Fibril-Mediated Toxicity

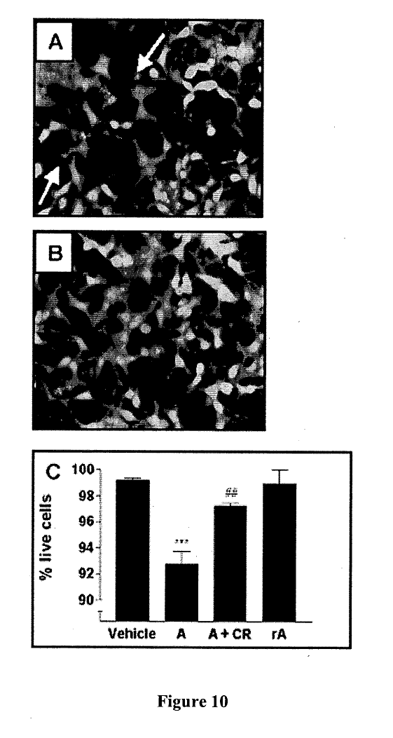

Protective effect of Congo red against amylin fibril-mediated toxicity in RINm5F cells (FIG. 10) Compounds which showed suppression of amyloid formation were further investigated for potential effects on amyloid-induced cytotoxicity in cultured RINm5F β cells. A. Representative fluorescence micrograph of RINm5F cells treated with 30 μM human amylin for 22 h and stained with calcein-AM and ethidium homodimer-1 to show live cells (green) and dead cells (red). Arrows denote examples of dead cells. B. A representative micrograph of RINm5F cells treated with 30 μM human amylin and 100 μM Congo red stained as above. C. The percentage of live cells was determined for cells treated for 22 h with vehicle; 30 μM human amylin; 30 μM human amylin in the presence of 100 μM Congo red; or 30 μM rat amylin. Experiments were repeated independently 10 times except for human amylin plus Congo red (A+CR) which was repeated five times. Error bars represent the s.e.m of live and dead cell counts over 6-12 fields per condition. Statistical significance was tested by one way ANOVA followed by pos-hoc analysis using Dunnett's test. ***p<0.001, ## p<0.01.

The compounds, neutral red, acridine orange, and tetracycline were cytotoxic to RINm5F βcells cells at the relative molar ratios which produced suppression of amyloid aggregation (low μM). Consequently, investigations were confined to Congo red which displayed nointrinsic cytotoxic effects under these experimental conditions. Results show incubation of RIN5mF cells with 30 μM amylin for 22 h resulted in a significant increase in cell death compared to the vehicle control (FIG. 10C). In contrast, rat amylin preparations under identical conditions were not cytotoxic. Dead cells are visible as red cells against a background of green live cells (FIG. 10A). Co-incubation of human amylin with a 3-fold molar excess of Congo red inhibited the cytotoxic effects of amylin (FIGS. 10B, 10C). Red staining of amylin fibrils (FIG. 10B) by Congo red can be seen in the background. These experiments were performed on three different commerical batches of amylin, and in each case significant protection was observed in the presence of Congo red.

In this study, the use of small polycyclic compounds as suppressors of amyloid formation was investigated. To explore potential structure/activity relationships, a representative series of small polycyclic compounds was selected on the basis of their aromatic ring topologies and sidechain components, or on previously reported inhibition of other amyloid-associated processes. Congo red is a conjugated biphenyl structure that is used routinely as a diagnostic non-specific amyloid stain in histopathology (Khurana et al., J. Biol. Chem. 276:22715-22721 (2001)). This compound has also been reported to inhibit fibrillar β-amyloid neurotoxicity in primary rat hippocampal cultures (Lorenzo et al., Proc. Natl. Acad. Sci. USA 91:12243-12247 (1994)), possibly through stabilization of the pre-amyloid monomer. See also Forloni G. et al., FEBS Lett. 487:404-407 (2001) regarding tetracycline, a four membered tetracene derivative, and the inhibition of β-amyloid formation and that the breakup of pre-formed β-amyloid. Chlorpromazine, a phenothiazine derivative, has been reported to reverse disease-forming prion plaques in scrapie infected mouse cell cultures and prolong cell survival (Korth et al., Proc. Natl. Acad. Sci. U.S.A 98:9836-9841 (2001)). Its isomer, thioflavin-T has been used extensively as a fluorescent probe to measure amyloid formation (Goldsbury C S, et al. (2000) J Struct. Biol. 130:217-231). Like chlorpromazine, methylene blue possesses a tricyclic core structure and is used clinically in the treatment of methemoglobinemia and as a dye to stain tissue in histopathology (Wright R O et al. (1999) Ann. Emerg. Med. 34:646-656). Neutral red, a tricyclic phenazine derivative, is used routinely as a specific fluorescent dye marker to identify and isolate pancreatic islets (Jager S et al. (1990 Eur. Surg. Res. 22:8-13). Acridine and acridine orange are examples of core and derivatised phenazine structures, respectively.

The present study shows that polycyclic compounds of the invention can suppress amyloid formation in vitro. A n aromatic phenazine core was sufficient to enable fibril binding, as demonstrated by the compound, acridine. Addition of two dimethylamine moieties at positions 2 and 8 to this core structure, yields acridine orange, which acted as a potent inhibitor of insoluble amyloid formation. Neutral red, a phenazine derivative, also inhibited amyloid formation, but at a significantly slower rate than acridine orange. In contrast, methylene blue, which is structurally identical to acridine orange except for a phenothiazine core, had no effect.

The striking differences between these tricyclic compounds on amyloid formation, as shown by the radioprecipitation studies, clearly indicate the existence of distinct structure relationships, which enable amyloid binding and an ability to suppress amyloid formation. Notably, while a core ring structure is sufficient for amyloid binding, presumably through aromatic π-π interactions (Gazit E FASEB J 16:77-83 (2002)), the presence of dimethylamine sidechains emanating from the ring are important for suppression of amyloid formation. Likewise, charged or non-charged phenothiazine derivatives were significantly less effective than respective phenazine derivatives. The importance of sidechain group interactions may also apply to extended biphenyl structures and tetracene derivatives as represented by Congo red and tetracycline, respectively.

The molecular mechanisms underlying the observed decrements in amyloid content are unknown. Unlike other amyloidoses, including Alzheimer P-amyloid and the prion protein, P rPc, where α-helix/β-strand-discordant stretches appear to be associated with amyloid formation (Kallberg Y et al. J. Biol. Chem. 276:12945-12950 (2001)), amyloid formation in the case of amylin likely proceeds via a pathway involving aggregation of relatively unfolded amyloid-forming regions. Although there is uncertainty over the precise identities of the folding assemblies involved, these aggregates lead to the formation of protofibrils composed of extended β-sheet structures with β-strand orientations perpendicular to the longitudinal axes. Of particular interest is the amyloidogenic region defined by residues 20-29, which includes the sequence, NFGAIL (Tenidis K et al., J. Mol. Biol. 295:1055-1071 (2000)). Substitutions within this region with prolyl residues at positions 25, 28, and 29, are sufficient to substantially decrease amyloid formation by the intact molecule. It is possible that the decrease in precipitable amyloid content observed by some of the polycyclic compounds investigated in this study are attributable to disruptive interactions within these amyloid-forming regions.

Preparations containing Congo red and human amylin were less cytotoxic to cultured islet β-cells than incubation with human amylin alone. Consequently, disruption of amyloid by polycyclic compounds may not necessarily be cytotoxic and may even be cytoprotective. Also, even subtle inhibitory effects on islet amyloid formation, in vivo, may be sufficient for compensatory endogenous clearance mechanisms to predominate and facilitate amyloid removal.

These findings demonstrate the utility of small polycyclic compounds as potential suppressors of islet amyloid formation.

What is claimed is:

1. A method of suppressing of cytotoxic protein conformers comprising administration of an effective amount of a three-membered ring polyacene, a substituted three-membered ring polyacene, a four-membered ring polyacene, a five-membered ring polyacene, a fused tetracyclic compound, or a fused ring and biphenyl compound.

2. A method of preventing an amyloid-associated disease comprising preventing protofibril formation and/or reduction of existing protofibril deposits.

3. A method according to claim 2 wherein said disease is prevented in a mammal.

4. A method according to claim 2 wherein said disease is prevented in a human being.

5. A method according to claim 2 wherein said disease is selected from the group comprising AL amyloidosis, amyloid A amyloidosis, familial transthyretin amyloidosis, Alzheimer's disease, prion diseases, or type II diabetes.

6. A method according to claim 2 wherein said disease is AL amyloidosis.

7. A method according to claim 2 wherein said disease is amyloid A amyloidosis.

8. A method according to claim 2 wherein said disease is familial transthyretin amyloidosis

9. A method according to claim 2 wherein said disease is Alzheimer's disease.

10. A method according to claim 2 wherein said disease is a prion disease.

11. A method according to claim 2 wherein said disease is type II diabetes.

12. A method according to claim 2 wherein said protofibril formation is prevented by administration of an effective amount of a three-membered ring polyacene, a substituted three-membered ring polyacene, a four-membered ring polyacene, a five-membered ring polyacene, a fused tetracyclic compound, or a fused ring and biphenyl compound.

13. A method according to claim 12 wherein said three-membered ring polyacene comprises anthracene, phenalene or phenanthrene.

14. A method according to claim 12 wherein said substituted three-membered ring polyacene comprises quinacrine, neutral red, chlorpromazine, acridine, acridine orange, methylene blue, or phenothiazine.

15. A method according to claim 12 wherein said four-membered ring polyacene comprises pyrene, chrysene, benz[a]anthracene, benz[m]anthracene or tetracene.

16. A method according to claim 12 wherein said five-membered ring polyacene comprises benzo[c]phenanthrene.

17. A method according to claim 12 wherein said fused tetracyclic compound comprises tetracycline or doxycycline.

18. A method according to claim 12 wherein said a fused ring and biphenyl compound comprises Congo red or chrysamine G or amaranth.

19. A method according to claim 12 wherein said three-membered ring polyacene is anthracene.

20. A method according to claim 12 wherein said three-membered ring polyacene is phenalene.

21. A method according to claim 12 wherein said three-membered ring polyacene is phenanthrene.

22. A method according to claim 13 wherein said substituted three-membered ring polyacene is quinacrine.

23. A method according to claim 13 wherein said substituted three-membered ring polyacene is neutral red.

24. A method according to claim 13 wherein said substituted three-membered ring polyacene is chlorpromazine.

25. A method according to claim 13 wherein said substituted three-membered ring polyacene is acridine.

26. A method according to claim 13 wherein said substituted three-membered ring polyacene is acridine orange.

27. A method according to claim 13 wherein said substituted three-membered ring polyacene is methylene blue.

28. A method according to claim 13 wherein said substituted three-membered ring polyacene is phenothiazine.

29. A method according to claim 14 wherein said four-membered ring polyacene is pyrene.

30. A method according to claim 14 wherein said four-membered ring polyacene is chrysene.

31. A method according to claim 14 wherein said four-membered ring polyacene is benz[a]anthracene.

32. A method according to claim 14 wherein said four-membered ring polyacene is benz[m]anthracene.

33. A method according to claim 14 wherein said four-membered ring polyacene is benzo[c]phenanthrene.

34. A method according to claim 14 wherein said four-membered ring polyacene is tetracene.

35. A method according to claim 16 wherein said fused tetracyclic compound is tetracycline.

36. A method according to claim 16 wherein said fused tetracyclic compound is doxycycline.

37. A method according to claim 17 wherein said fused ring and biphenyl compound is congo red.

38. A method according to claim 17 wherein said fused ring and biphenyl compound is chrysamine G.

39. A method according to claim 12 wherein said administered compound is selected from a group consisting essentially of anthracene, phenanthrene, quinacrine, neutral red, chlorpromazine, acridine, acridine orange, methylene blue, phenodiazine, phenothiazine, tetracycline, doxycycline, congo red, pyrene, chrysene, benz[a]anthracene, benz[m]anthracene, benzo[c]phenanthrene and tetracene.

40. A method according to claim 12 wherein said protofibril formation is prevented by administration of a combination of a three-membered ring polyacene, a substituted three-membered ring polyacene, a four-membered ring polyacene, a five-membered ring polyacene, a fused tetracyclic compound, and/or a fused ring and biphenyl compound.

41. A method of preventing or inhibiting protofibril formation comprising administering an effective amount of three-membered ring polyacene, a substituted three-membered ring polyacene, a four-membered ring polyacene, a five-membered ring polyacene, a fused tetracyclic compound, or a fused ring and biphenyl compound.

42. A method according to claim 41 wherein said three-membered ring polyacene, a substituted three-membered ring polyacene, a four-membered ring polyacene, a five-membered ring polyacene, a fused tetracyclic compound, or a fused ring and biphenyl compound comprises anthracene, phenanthrene, quinacrine, neutral red, chlorpromazine, acridine, acridine orange, methylene blue, phenodiazine, phenothiazine, tetracycline, doxycycline, congo red, pyrene, chrysene, benz[a]anthracene, benz[m]anthracene, benzo[c]phenanthrene or tetracene.

43. A method of ameliorating an amyloid-associated disease comprising preventing protofibril formation.

44. A method of preventing islet β-cell death comprising preventing protofibril formation.

45. A method of preventing amyloid-associated disease in a human susceptible to said disease comprising preventing protofibril formation.

46. A method of preventing the transition from soluble amylin to insoluble amylin comprising administering an effective amount of an appropriate of a three-membered ring polyacene, a substituted three-membered ring polyacene, a four-membered ring polyacene, a five-membered ring polyacene, a fused tetracyclic compound, or a fused ring and biphenyl compound.

47. A method of preventing cytotoxic β-conformer formation comprising administering an effective amount of a three-membered ring polyacene, a substituted three-membered ring polyacene, a four-membered ring polyacene, a five-membered ring polyacene, a fused tetracyclic compound, or a fused ring and biphenyl compound.

48. A method of preventing or ameliorating diseases associated with amyloidosis comprising administering an effective amount of a three-membered ring polyacene, a substituted three-membered ring polyacene, a four-membered ring polyacene, a five-membered ring polyacene, a fused tetracyclic compound, or a fused ring and biphenyl compound prior to protofibril formation.

49. A method of screening for a compound or compounds effective to downregulate the β-conformer of amylin, for example, human amylin, comprising or including the steps of
(i) administration of the compound or compounds to a preparation of amylin with or without β-conformer present;
(ii) identifying and/or determining the level or levels of β-conformer, thereby to determine effectiveness.

50. A method of identifying compounds that can block toxicity normally associated with amyloid resulting from the transition from soluble amylin to insoluble amylin or the formation of protofibrils comprising administration of the compound or compounds to a preparation of amylin and observing β-conformer formation.

51. A method of screening for a compound or compounds effective to downregulate the β-conformer of human amylin comprising or including the steps of
(i) administration of the compound or compounds to a preparation of amylin with or without β-conformer present;
(ii) identifying and/or determining the level or levels of β-conformer, thereby to determine effectiveness.

52. A method of claim 51 wherein the compound(s) is (or are) polycyclic.

53. A method according to claim 51 wherein the β-conformer is identified and/or the level or levels of β-conformer is determined by physical separation, purification, isolation, and/or precipitation.

54. A method according to claim 51 wherein the amylin is at least in part labeled and/or tagged.

55. A method according to claim 51 wherein the label or tag comprises any one or more of the following: radioisotope, fluorescent tag, antibody, optically detectable, or enzymatic.

56. A method according to claim 51 wherein the β-conformer is identified and/or the level or levels of β-conformer is determined by the binding or release of an affinity label.

57. The method according to claim 51 wherein the affinity label is thioflavin-T.

58. The method according to claim 51 wherein the affinity label is heparin or a functional variant thereof.

59. A method according to claim 51 wherein the β-conformer is identified and/or the level or levels of β-conformer is determined by circular dichroism.

60. A method according to claim 51 wherein the β-conformer is identified and/or the level or levels of β-conformer is determined by electron microscopy.

61. A compound identified by the method of claim 51.

62. A method of screening for a compound or compounds effective to inhibit the transition and/or decrease the rate of the transition of human amylin from a conformation comprising or including random coil and/or α-helix to a conformation comprising or including β-sheet, wherein said method comprises or includes the steps of
(i) administration of the compound or compounds to a preparation of amylin with or without β-conformer present;
(ii) identifying and/or determining the level or levels of β-conformer, thereby to determine effectiveness.

63. A method of claim 62 wherein the compound(s) is (or are) polycyclic.

64. A method according to claim 62 wherein the β-conformer is identified and/or the level or levels of β-conformer is determined by physical separation, purification, isolation, and/or precipitation.

65. A method according to claim 62 wherein the amylin is at least in part labelled and/or tagged.

66. A method according to claim 62 wherein the label or tag comprises any one or more of the following: radioisotope, fluorescent tag, antibody, optically detectable, or enzymatic.

67. A method according to claim 62 wherein the β-conformer is identified and/or the level or levels of β-conformer is determined by the binding or release of an affinity label.

68. The method according to claim 62 wherein the affinity label is thioflavin-T.

69. The method according to claim 62 wherein the affinity label is heparin or a functional variant thereof.

70. A method according to claim 62 wherein the β-conformer is identified and/or the level or levels of β-conformer is determined by circular dichroism.

71. A method according to claim 62 wherein the β-conformer is identified and/or the level or levels of β-conformer is determined by electron microscopy.

72. A compound identified by the method of claim 62.

73. A method of screening for a compound or compounds effective for the suppression of islet β-cell degeneration and/or reversal of disease state comprising or including the steps of
(i) administration of the compound or compounds to a preparation of amylin with or without β-conformer present;
(ii) identifying and/or determining the level or levels of β-conformer, thereby to determine effectiveness.

74. A method of claim 73 wherein the compound(s) is (or are) polycyclic.

75. A method according to claim 73 wherein the β-conformer is identified and/or the level or levels of β-conformer is determined by physical separation, purification, isolation, and/or precipitation.

76. A method according to claim 73 wherein the amylin is at least in part labelled and/or tagged.

77. A method according to claim 73 wherein the label or tag comprises any one or more of the following: radioisotope, fluorescent tag, antibody, optically detectable, or enzymatic.

78. A method according to claim 73 wherein the β-conformer is identified and/or the level or levels of β-conformer is determined by the binding or release of an affinity label.

79. The method according to claim 73 wherein the affinity label is thioflavin-T.

80. The method according to claim 73 wherein the affinity label is heparin or a functional variant thereof.

81. A method according to claim 73 wherein the β-conformer is identified and/or the level or levels of β-conformer is determined by circular dichroism.

82. A method according to claim 73 wherein the β-conformer is identified and/or the level or levels of β-conformer is determined by electron microscopy. A compound identified by the method of claim 73.

83. A method of screening for a compound or compounds effective for the suppression of islet β-cell degeneration and/or reversal of disease state comprising or including the steps of
(i) administration of the compound or compounds to a cell or cells capable of producing the β-conformer of human amylin;
(ii) identifying and/or determining the level or levels of β-conformer within or extracellular to the cell or cells, thereby to determine effectiveness;
(iii) co-administration of compound(s) with exogenous human amylin which is capable of forming cytotoxic β-conformer(s) to cell(s) to determine effects on cell viability by cell death assays.

84. A method of claim 83 wherein the compound(s) is (or are) polycyclic.

85. A method according to claim 83 wherein the β-conformer is identified and/or the level or levels of β-conformer is determined by physical separation, purification, isolation, and/or precipitation.

86. A method according to claim 83 wherein the amylin is at least in part labelled and/or tagged.

87. A method according to claim 83 wherein the label or tag comprises any one or more of the following: radioisotope, fluorescent tag, antibody, optically detectable, or enzymatic.

88. A method according to claim 83 wherein the β-conformer is identified and/or the level or levels of β-conformer is determined by the binding or release of an affinity label.

89. The method according to claim 83 wherein the affinity label is thioflavin-T.

90. The method according to claim 83 wherein the affinity label is heparin or a functional variant thereof.

91. A method according to claim 83 wherein the β-conformer is identified and/or the level or levels of β-conformer is determined by circular dichroism.

92. A method according to claim 83 wherein the β-conformer is identified and/or the level or levels of β-conformer is determined by electron microscopy.

93. A compound identified by the method of claim 83.

94. A method of screening for a compound or compounds effective to downregulate β-conformer of human amylin comprising or including the steps of
(i) administration of the compound or compounds to a cell or cells capable of producing the β-conformer of human amylin;
(ii) identifying and/or determining the level or levels of β-conformer within or extracellular to the cell or cells, thereby to determine effectiveness;
(iii) co-administration of compound(s) with exogenous human amylin which is capable of forming cytotoxic β-conformer(s) to cell(s) to determine effects on cell viability by cell death assays.

95. A method of claim 94 wherein the compound(s) is (or are) polycyclic.

96. A method according to claim 94 wherein the β-conformer is identified and/or the level or levels of β-conformer is determined by physical separation, purification, isolation, and/or precipitation.

97. A method according to claim 94 wherein the amylin is at least in part labelled and/or tagged.

98. A method according to claim 94 wherein the label or tag comprises any one or more of the following: radioisotope, fluorescent tag, antibody, optically detectable, or enzymatic.

99. A method according to claim 94 wherein the β-conformer is identified and/or the level or levels of β-conformer is determined by the binding or release of an affinity label.

100. The method according to claim 94 wherein the affinity label is thioflavin-T.

101. The method according to claim 94 wherein the affinity label is heparin or a functional variant thereof.

102. A method according to claim 94 wherein the β-conformer is identified and/or the level or levels of β-conformer is determined by circular dichroism.

103. A method according to claim 94 wherein the β-conformer is identified and/or the level or levels of β-conformer is determined by electron microscopy.

104. A compound identified by the method of claim 94.

105. A method of screening for a compound or compounds effective to inhibit the transition and/or decrease the rate of the transition of human amylin from a conformation comprising or including random coil and/or a-helix to a conformation comprising or including β-sheet, wherein said method comprises or includes the steps of (i) administration of the compound or compounds to a cell or cells capable of producing the β-conformer of human amylin;
(ii) identifying and/or determining the level or levels of β-conformer within or extracellular to the cell or cells, thereby to determine effectiveness;
(iii) co-administration of compound(s) with exogenous human amylin which is capable of forming cytotoxic β-conformer(s) to cell(s) to determine effects on cell viability by cell death assays.

106. A method of claim 105 wherein the compound(s) is (or are) polycyclic.

107. A method according to claim 105 wherein the β-conformer is identified and/or the level or levels of β-conformer is determined by physical separation, purification, isolation, and/or precipitation.

108. A method according to claim 105 wherein the amylin is at least in part labelled and/or tagged.

109. A method according to claim 105 wherein the label or tag comprises any one or more of the following: radioisotope, fluorescent tag, antibody, optically detectable, or enzymatic.

110. A method according to claim 105 wherein the β-conformer is identified and/or the level or levels of β-conformer is determined by the binding or release of an affinity label.

111. The method according to claim 105 wherein the affinity label is thioflavin-T.

112. The method according to claim 105 wherein the affinity label is heparin or a functional variant thereof.

113. A method according to claim 105 wherein the β-conformer is identified and/or the level or levels of α-conformer is determined by circular dichroism.

114. A method according to claim 105 wherein the β-conformer is identified and/or the level or levels of β-conformer is determined by electron microscopy.

115. A compound identified by the method of claim 105.

116. A method of screening for a compound or compounds effective to downregulate the β-conformer of human amylin comprising or including the steps of
(i) administration of the compound or compounds to a cell or cells capable of producing the β-conformer of human amylin;
(ii) characterising and/or determining the activation and/or upregulation of cellular markers of cell death as an indication of ineffectiveness, thereby to determine effectiveness.

117. A method of claim 116 wherein the compound(s) is (or are) polycyclic.

118. The method according to claim 116 wherein the cellular marker(s) is (or are) apoptotic markers.

119. The method according to claim 116 wherein the cellular marker(s) is (or are) necrotic markers.

120. The method according to claim 116 wherein the cellular marker(s) is (or are) selected from any one or more of the following: caspase8, caspase3, cjun, JNK, p21, AF1, p53.

121. The method according to claim 116 wherein the characterisation or determination utilises at least one of the mRNA species capable of encoding one or more of the following: caspase8, caspase3, cjun, JNK, p21, WAF1, p53.

122. The method according to claim 116 wherein the characterisation or determination utilises RT-PCR, Northern analysis, hybridisation, or microarray.

123. The method according to claim 116 wherein the characterisation or determination utilises one or more polypeptide of the following group: caspase8, caspase3, cjun, JNK, p21 WAF1, p53.

124. A compound identified by the method of claim 116.
